SEQUENCE LISTING (68 sequences)

SEQ ID NO: 1 - protein, 15 aa, Arachis hypogea
  REGION 1..15: note = ara h 2.008 peptide
WELQGDRRCQSQLER

SEQ ID NO: 2 - protein, 15 aa, Arachis hypogea
  REGION 1..15: note = ara h 3.100 peptide
EYDEDEYEYDEEDRR

SEQ ID NO: 3 - protein, 619 aa, Arachis hypogea
  REGION 1..619: note = Ara h1
MRGRVSPLMLLLGILVLASVSATHAKSSPYQKKTENPCAQRCLQSCQQEPDDLKQKACESRCTKLEYDPRCVYDPRGHTGTTNQRSPPGERTRGRQPGDYDDDRRQPRREEGGRWGPAGPREREREEDWRQPREDWRRPSHQQPRKIRPEGREGEQEWGTPGSHVREETSRNNPFYFPSRRFSTRYGNQNGRIRVLQRFDQRSRQFQNLQNHRIVQIEAKPNTLVLPKHADADNILVIQQGQATVTVANGNNRRALILTRAMHSESHPFHFLHLDDMTPELRVAKSHAVNTPGQFEDFFPASSRDQSSYLQGFSRNTLEAAFNAEFNEIRRVLLEENAGGEQEERGQRRWSTRSSENNEGVIVEVSKEHVEELTKHAKSVSKKGSEEEGDITNPINLREGEPDLSDNFGRLFEVKPDKKNPQLQDLDMMLTCVEIKEGALMLPHFNSKAMVIVVINKGTGNLELVAVRKEQQQRGRREQEWEEEEEDEEEEGSNREVRRYTARLKEGDVFIMPAAHPVAINASSELHLLGFGINAENNHRIFLAGDKDNVVDQIEKQAKDLAFPGSGEQVEKLIKNQRESHFVSARPQSQSPSSPEKEDQEEENQGGKGPLLSILKAFN

SEQ ID NO: 4 - protein, 172 aa, Arachis hypogea
  REGION 1..172: note = Ara h2
MAKLTILVALALFLLAAHASARQQWELQGDRRCQSQLERANLRPCEQHLMQKIQRDEDSYGRDPYSPSQDPYSPSQDPDRRDPYSPSPYDRRGAGSSQHQERCCNELNEFENNQRCMCEALQQIMENQSDRLQGRQQEQQFKRELRNLPQQCGLRAPQRCDLEVESGGRDRY

SEQ ID NO: 5 - protein, 512 aa, Arachis hypogea
  REGION 1..512: note = Ara h3
MAKLLALSLCFCVLVLGASSVTFRQGGEENECQFQRLNAQRPDNRIESEGGYIETWNPNNQEFQCAGVALSRTVLRRNALRRPFYSNAPLEIYVQQGSGYFGLIFPGCPSTYEEPAQEGRRYQSQKPSRRFQVGQDDPSQQQQDSHQKVHRFDEGDLIAVPTGVAFWMYNDEDTDVVTVTLSDTSSIHNQLDQFPRRFYLAGNQEQEFLRYQQQQGSRPHYRQISPRVRGDEQENEGSNIFSGFAQEFLQHAFQVDRQTVENLRGENEREEQGAIVTVKGGLRILSPDEEDESSRSPPNRREEFDEDRSRPQQRGKYDENRRGYKNGIEETICSASVKKNLGRSSNPDIYNPQAGSLRSVNELDLPILGWLGLSAQHGTIYRNAMFVPHYTLNAHTIVVALNGRAHVQVVDSNGNRVYDEELQEGHVLVVPQNFAVAAKAQSENYEYLAFKTDSRPSIANQAGENSIIDNLPEEVVANSYRLPREQARQLKNNNPFKFFVPPFDHQSMREVA

SEQ ID NO: 6 - protein, 15 aa, Arachis hypogea
  REGION 1..15: note = ara h 1.008 peptide
ATHAKSSPYQKKTEN

SEQ ID NO: 7 - protein, 15 aa, Arachis hypogea
  REGION 1..15: note = ara h 1.015 peptide
LQSCQQEPDDLKQKA

SEQ ID NO: 8 - protein, 15 aa, Arachis hypogea
  REGION 1..15: note = ara h 1.021 peptide
RCTKLEYDPRCVYDP

SEQ ID NO: 9 - protein, 15 aa, Arachis hypogea
  REGION 1..15: note = ara h 1.022 peptide
KLEYDPRCVYDPRGH

SEQ ID NO: 10 - protein, 15 aa, Arachis hypogea
  REGION 1..15: note = ara h 1.025 peptide
YDPRGHTGTTNQRSP

SEQ ID NO: 11 - protein, 15 aa, Arachis hypogea
  REGION 1..15: note = ara h 1.029 peptide
RSPPGERTRGRQPGD

SEQ ID NO: 12 - protein, 15 aa, Arachis hypogea
  REGION 1..15: note = ara h 1.030 peptide
PGERTRGRQPGDYDD

SEQ ID NO: 13 - protein, 15 aa, Arachis hypogea
  REGION 1..15: note = ara h 1.033 peptide
PGDYDDDRRQPRREE

SEQ ID NO: 14 - protein, 15 aa, Arachis hypogea
  REGION 1..15: note = ara h 1.035 peptide
DRRQPRREEGGRWGP

SEQ ID NO: 15 - protein, 15 aa, Arachis hypogea
  REGION 1..15: note = ara h 1.040 peptide
AGPREREREEDWRQP

SEQ ID NO: 16 - protein, 15 aa, Arachis hypogea
  REGION 1..15: note = ara h 1.041 peptide
REREREEDWRQPRED

SEQ ID NO: 17 - protein, 15 aa, Arachis hypogea
  REGION 1..15: note = ara h 1.044 peptide
RQPREDWRRPSHQQP

SEQ ID NO: 18 - protein, 15 aa, Arachis hypogea
  REGION 1..15: note = ara h 1.045 peptide
REDWRRPSHQQPRKI

SEQ ID NO: 19 - protein, 15 aa, Arachis hypogea
  REGION 1..15: note = ara h 1.047 peptide
PSHQQPRKIRPEGRE

SEQ ID NO: 20 - protein, 15 aa, Arachis hypogea
  REGION 1..15: note = ara h 1.050 peptide
RPEGREGEQEWGTPG

SEQ ID NO: 21 - protein, 15 aa, Arachis hypogea
  REGION 1..15: note = ara h 1.056 peptide
REETSRNNPFYFPSR

SEQ ID NO: 22 - protein, 15 aa, Arachis hypogea
  REGION 1..15: note = ara h 1.058 peptide
NNPFYFPSRRFSTRY

SEQ ID NO: 23 - protein, 15 aa, Arachis hypogea
  REGION 1..15: note = ara h 1.090 peptide
SGFISYILNRHDNQN

SEQ ID NO: 24 - protein, 15 aa, Arachis hypogea
  REGION 1..15: note = ara h 1.097 peptide
SMPVNTPGQFEDFFP

SEQ ID NO: 25 - protein, 15 aa, Arachis hypogea
  REGION 1..15: note = ara h 1.103 peptide
RDQSSYLQGFSRNTL

SEQ ID NO: 26 - protein, 15 aa, Arachis hypogea
  REGION 1..15: note = ara h 1.130 peptide
SEEEGDITNPINLRE

SEQ ID NO: 27 - protein, 15 aa, Arachis hypogea
  REGION 1..15: note = ara h 1.131 peptide
EGDITNPINLREGEP

SEQ ID NO: 28 - protein, 15 aa, Arachis hypogea
  REGION 1..15: note = ara h 1.137 peptide
NNFGKLFEVKPDKKN

SEQ ID NO: 29 - protein, 15 aa, Arachis hypogea
  REGION 1..15: note = ara h 1.167 peptide
RYTARLKEGDVFIMP

SEQ ID NO: 30 - protein, 15 aa, Arachis hypogea
  REGION 1..15: note = ara h 1.170 peptide
DVFIMPAAHPVAINA

SEQ ID NO: 31 - protein, 15 aa, Arachis hypogea
  REGION 1..15: note = ara h 1.173 peptide
PVAINASSELHLLGF

SEQ ID NO: 32 - protein, 15 aa, Arachis hypogea
  REGION 1..15: note = ara h 1.176 peptide
LHLLGFGINAENNHR

SEQ ID NO: 33 - protein, 15 aa, Arachis hypogea
  REGION 1..15: note = ara h 1.179 peptide
AENNHRIFLAGDKDN

SEQ ID NO: 34 - protein, 15 aa, Arachis hypogea
  REGION 1..15: note = ara h 1.180 peptide
NHRIFLAGDKDNVID

SEQ ID NO: 35 - protein, 15 aa, Arachis hypogea
  REGION 1..15: note = ara h 1.184 peptide
VIDQIEKQAKDLAFP

SEQ ID NO: 36 - protein, 15 aa, Arachis hypogea
  REGION 1..15: note = ara h 1.186 peptide
KQAKDLAFPGSGEQV

SEQ ID NO: 37 - protein, 15 aa, Arachis hypogea
  REGION 1..15: note = ara h 1.187 peptide
KDLAFPGSGEQVEKL

SEQ ID NO: 38 - protein, 15 aa, Arachis hypogea
  REGION 1..15: note = ara h 1.194 peptide
SHFVSARPQSQSQSP

SEQ ID NO: 39 - protein, 15 aa, Arachis hypogea
  REGION 1..15: note = ara h 1.203 peptide
QEEENQGGKGPLLSI

SEQ ID NO: 40 - protein, 15 aa, Arachis hypogea
  REGION 1..15: note = ara h 2.005 peptide
AAHASARQQWELQGD

SEQ ID NO: 41 - protein, 15 aa, Arachis hypogea
  REGION 1..15: note = ara h 2.010 peptide
RRCQSQLERANLRPC

SEQ ID NO: 42 - protein, 15 aa, Arachis hypogea
  REGION 1..15: note = ara h 2.014 peptide
RPCEQHLMQKIQRDE

SEQ ID NO: 43 - protein, 15 aa, Arachis hypogea
  REGION 1..15: note = ara h 2.017 peptide
KIQRDEDSYERDPYS

SEQ ID NO: 44 - protein, 15 aa, Arachis hypogea
  REGION 1..15: note = ara h 2.018 peptide
RDEDSYERDPYSPSQ

SEQ ID NO: 45 - protein, 15 aa, Arachis hypogea
  REGION 1..15: note = ara h 2.019 peptide
DSYERDPYSPSQDPY

SEQ ID NO: 46 - protein, 15 aa, Arachis hypogea
  REGION 1..15: note = ara h 2.021 peptide
PYSPSQDPYSPSPYD

SEQ ID NO: 47 - protein, 15 aa, Arachis hypogea
  REGION 1..15: note = ara h 2.030 peptide
CCNELNEFENNQRCM

SEQ ID NO: 48 - protein, 15 aa, Arachis hypogea
  REGION 1..15: note = ara h 2.031 peptide
ELNEFENNQRCMCEA

SEQ ID NO: 49 - protein, 15 aa, Arachis hypogea
  REGION 1..15: note = ara h 2.036 peptide
LQQIMENQSDRLQGR

SEQ ID NO: 50 - protein, 15 aa, Arachis hypogea
  REGION 1..15: note = ara h 2.037 peptide
IMENQSDRLQGRQQE

SEQ ID NO: 51 - protein, 15 aa, Arachis hypogea
  REGION 1..15: note = ara h 2.038 peptide
NQSDRLQGRQQEQQF

SEQ ID NO: 52 - protein, 15 aa, Arachis hypogea
  REGION 1..15: note = ara h 2.040 peptide
QGRQQEQQFKRELRN

SEQ ID NO: 53 - protein, 15 aa, Arachis hypogea
  REGION 1..15: note = ara h 2.043 peptide
KRELRNLPQQCGLRA

SEQ ID NO: 54 - protein, 15 aa, Arachis hypogea
  REGION 1..15: note = ara h 2.045 peptide
LPQQCGLRAPQRCDL

SEQ ID NO: 55 - protein, 15 aa, Arachis hypogea
  REGION 1..15: note = ara h 3.018 peptide
LRRNALRRPFYSNAP

SEQ ID NO: 56 - protein, 15 aa, Arachis hypogea
  REGION 1..15: note = ara h 3.030 peptide
HYEEPHTQGRRSQSQ

SEQ ID NO: 57 - protein, 15 aa, Arachis hypogea
  REGION 1..15: note = ara h 3.031 peptide
EPHTQGRRSQSQRPP

SEQ ID NO: 58 - protein, 15 aa, Arachis hypogea
  REGION 1..15: note = ara h 3.037 peptide
QGEDQSQQQRDSHQK

SEQ ID NO: 59 - protein, 15 aa, Arachis hypogea
  REGION 1..15: note = ara h 3.060 peptide
NTEQEFLRYQQQSRQ

SEQ ID NO: 60 - protein, 15 aa, Arachis hypogea
  REGION 1..15: note = ara h 3.068 peptide
PYSPQSQPRQEEREF

SEQ ID NO: 61 - protein, 15 aa, Arachis hypogea
  REGION 1..15: note = ara h 3.079 peptide
EGGNIFSGFTPEFLE

SEQ ID NO: 62 - protein, 15 aa, Arachis hypogea
  REGION 1..15: note = ara h 3.080 peptide
NIFSGFTPEFLEQAF

SEQ ID NO: 63 - protein, 15 aa, Arachis hypogea
  REGION 1..15: note = ara h 3.092 peptide
AIVTVRGGLRILSPD

SEQ ID NO: 64 - protein, 15 aa, Arachis hypogea
  REGION 1..15: note = ara h 3.093 peptide
TVRGGLRILSPDRKR

SEQ ID NO: 65 - protein, 15 aa, Arachis hypogea
  REGION 1..15: note = ara h 3.102 peptide
YEYDEEDRRRGRGSR

SEQ ID NO: 66 - protein, 15 aa, Arachis hypogea
  REGION 1..15: note = ara h 3.152 peptide
IANLAGENSVIDNLP

SEQ ID NO: 67 - protein, 15 aa, Arachis hypogea
  REGION 1..15: note = ara h 3.162 peptide
RQLKNNNPFKFFVPP

SEQ ID NO: 68 - protein, 145 aa, Arachis hypogea
  REGION 1..145: note = Ara h 6
MAKSTILVALLALVLVAHASAMRRERGRQGDSSSCERQVDRVNLKPCEQHIMQRIMGEQEQYDSYDIRSTRSSDQQQRCCDELNEMENTQRCMCEALQQIMENQCDRLQDRQMVQQFKRELMNLPQQCNFRAPQRCDLDVSGGRC

(19) United States
(12) Patent Application Publication
(10) Pub. No.: US 2024/0255526 A1
(43) Pub. Date: Aug. 1, 2024

(54) Assays For Determining Severity Of Peanut Allergies

(71) Applicant: AllerGenis, Inc., Wynnewood, PA (US)

(72) Inventors: Paul Kearney, Wynnewood, PA (US); Heather Butler, Wynnewood, PA (US); Clive Hayward, Wynnewood, PA (US)

(21) Appl. No.: 18/428,273

(22) Filed: Jan. 31, 2024

Related U.S. Application Data
(60) Provisional application No. 63/442,633, filed on Feb. 1, 2023.

(51) Int. Cl. G01N 33/68

(52) U.S. Cl. CPC G01N 33/6893; G01N 33/6854; G01N 2333/415; G01N 2800/24

(57) ABSTRACT

Methods for determining a personalized reaction severity risk for subjects that are allergic to peanuts are described herein.

### REFERENCE TO SEQUENCE LISTING

This application includes a Sequence Listing filed electronically as an XML file named 896983049SEQ, created on Jan. 29, 2024 with a size of 86,902 bytes. The Sequence Listing is incorporated herein by reference.

### FIELD

The present disclosure is directed, in part, to methods for determining a personalized severity risk for subjects that are allergic to peanuts.

### BACKGROUND

Food allergies are a common problem among adults and children, and symptoms may range from mild oral pruritus to potentially life-threatening anaphylactic shock. Food allergies are currently diagnosed by skin prick testing or oral provocation, and measurement of serum levels of specific IgE and, in some cases, other serum antibodies, such as IgG4. Although these tests indicate the likelihood of clinical reactivity, they do not distinguish the different phenotypes of food allergy or provide prognostic information. Current allergy tests also involve some level of risk to the patient. The relationship between current IgE testing and the actual clinical sensitivity of the patient is a weak one that is usually defined as a combination of reaction severity and the amount of allergen that provokes a reaction. Another limitation of current testing is the inability to determine whether or not pediatric patients will outgrow the allergy during childhood. In this case, there is a positive but weak correlation between specific IgE level and the duration of clinical allergy.

More recently, it has been suggested that clinical reactivity to food allergens may correlate better with allergen-specific IgE on the epitope recognition level. It has been reported that patients with persistent or more severe allergic reactions recognize larger numbers of IgE epitopes, suggesting epitope mapping as an additional tool for allergy diagnosis and prediction. Spot membrane-based immunoassays have been used for epitope mapping. In this system, peptides are synthesized on the membrane and incubated with the patient's sera. The process requires a large number of peptides and is, therefore, error prone, time consuming, labor intensive, and expensive. Immunoassays in this format also require a large volume of patient serum.

The marked heterogeneity of clinical presentations for food allergy poses a challenge to successful management and treatment. Sensitive and specific biomarkers for determination of food allergy endotypes, risk of developing allergies, reaction severity, and prognosis with treatment are components in the path toward precision medicine (Sicherer et al., J. Allergy Clin. Immunol., 2015, 135, 357-67). In the past decade, there have been a number of studies evaluating the efficacy of oral immunotherapy (OIT) for the treatment of persistent food allergies (Wood et al., J. Allergy Clin. Immunol., 2016, 137, 1103-1110). In peanut allergy, OIT has been shown to have acceptable safety profile and demonstrated clinical benefit (Bird et al., J. Allergy Clin. Immunol. Pract., 2017, 5, 335-344). Despite the improvement in clinical reactivity, OIT has been associated with significant adverse effects, with some experiencing anaphylaxis and 15% to 20% forced to discontinue therapy because of adverse reactions (Bird et al., J. Allergy Clin. Immunol. Pract., 2017, 5, 335-344; Keet et al., J. Allergy Clin. Immunol., 2012, 129, 448-455; Longo et al., J. Allergy Clin. Immunol., 2008, 121, 343-7; Meglio et al., Pediatr. Allergy Immunol., 2008, 19, 412-419; Skripak et al., J. Allergy Clin. Immunol., 2008, 122, 1154-60; and Staden et al., Allergy, 2007, 62, 1261-1269). In addition to adverse reactions, the response to OIT is typically not sustained once therapy is discontinued, i.e. patients are temporarily desensitized to allergens but do not achieve tolerance (Wood et al., J. Allergy Clin. Immunol., 2016, 137, 1103-1110; Burks et al., N. Engl. J. Med., 2012, 367, 233-243; Burks et al., J. Allergy Clin. Immunol., 2008, 121, 1344-1350; Burks, Arb. Paul Ehrlich Inst. Bundesinstitut Impfstoffe Biomed Arzneim Langen Hess, 2013, 97, 122-123; Gorelik et al., J. Allergy Clin. Immunol., 2015, 135, 1283-1292; and Keet et al., J. Allergy Clin. Immunol., 2013, 132, 737-739). These therapeutic approaches will benefit from a diagnostic and prognostic test which will help patients and their doctors understand the severity of the disease upon entry into therapy, monitor a patient while on therapy to assess progress or onset of an adverse reaction before it occurs, and track patient status once treatment is discontinued.

The production of IgE antibodies against peanut proteins is central to the pathogenesis of peanut allergy. Although predictive curves have been generated to identify peanut specific IgE concentrations which are 95% predictive of clinical reactivity, peanut-IgE is poorly predictive at lower IgE levels, and at higher levels the readout is only binary and is therefore difficult to use to help assess the safety or efficacy of therapy. This may be due to measurement of IgE antibodies against components of peanut which are not clinically relevant. IgE against Ara h 2 predicts clinical reactivity to peanut (Lieberman et al., J. Allergy Clin. Immunol. Pract., 2013, 1, 75-82) but there is a great deal of clinical heterogeneity across individuals with similar levels of Ara h 2. Peptide microarrays comprised of overlapping peptides covering the entire sequential epitope repertoire of major allergens have been developed to measure the epitope-specific immunoglobulin response (Lin et al., J. Allergy Clin. Immunol., 2009, 124, 315-22; and Lin et al., J. Allergy Clin. Immunol., 2012, 129, 1321-1328). The number of peanut epitopes in Ara h 1, 2 and 3 which bind to IgE is predictive of reaction severity (Flinterman et al., J. Allergy Clin. Immunol., 2008, 121, 737-743). As a component-resolved diagnostic methodology (ImmunoCAP), the presence of sIgE to peanut, Ara h1, Ara h2, and Ara h3 is indicative of a “true” peanut allergy and a high risk of severe reactions (e.g., levels of sIgE≥0.35 kU_{A}/L show 75-95% PPV, 90% NPV in diagnosing allergy; Klemans et al., J. Allergy Clin. Immunol., 2013, 131, 157-163).

In determining whether a subject is allergic to a given oral allergen, physicians typically administer a dose of the allergen to the subject in a small amount with an increase in that amount upon subsequent administrations. The allergen amount that induces a medically unacceptable response is known as an eliciting dose (ED) or a reactive dose (RD) while the amount of allergen in the previous administration to the administration of the ED or RD is known as the tolerated dose (TD). The cumulative reactive dose (CRD) is the arithmetic sum of the amounts of the allergen administered to the subject while the cumulated tolerated dose (CTD) is the sum of the amounts of the allergen administered to the subject including the amount of the TD but excluding the amount of the ED or RD. CRDs are frequently determined in double-blind, placebo-controlled food challenge (DBPCFC) clinical trials that are frequently conducted in connection with the PRACTALL dosing protocol (see, Sampson et al., J. Allergy Clin. Immunol., 2012, 130, 1260-74 (“Sampson”)). However, determination of CRDs currently requires administration of allergen to subjects that causes at best discomfort and at worse a full anaphylactic response. Accordingly, there is a long felt but unmet need for methods to determine CRDs without the potentially hazardous administration of allergens and to determine the association with a severe reaction. To date, beyond oral food challenges, there is no stable and low-risk blood test that can predict a severe reaction risk for patients.

### SUMMARY

The present disclosure provides methods for determining the risk of anaphylaxis in a subject allergic to peanuts, the methods comprising: a) determining a threshold cumulative reactive dose of a peanut peptide for the subject comprising: i) contacting at least one first peanut peptide comprising the amino acid sequence WELQGDRRCQSQLER (SEQ ID NO:1), or an amino acid sequence comprising SEQ ID NO:1 but having one to four conservative amino acid substitutions therein, coupled to at least one first solid support with at least one first biological sample obtained from the subject, wherein the contacting is under conditions sufficient to permit binding of at least one allergy associated immunoglobulin (AAI-1) present in the at least one first biological sample to the at least one first peanut peptide to form at least one AAI-1-peptide-solid support complex; ii) contacting at least one second peanut peptide comprising the amino acid sequence EYDEDEYEYDEEDRR (SEQ ID NO:2), or an amino acid sequence comprising SEQ ID NO:2 but having one to four conservative amino acid substitutions therein, coupled to at least one second solid support with at least one second biological sample obtained from the subject, wherein the contacting is under conditions sufficient to permit binding of at least one second allergy associated immunoglobulin (AAI-2) present in the at least one second biological sample to the at least one second peanut peptide to form at least one AAI-2-peptide-solid support complex; iii) contacting the at least one AAI-1-peptide-solid support complex with at least one AAI-1-specific labeling reagent under conditions sufficient to permit binding of the at least one AAI-1 specific labeling reagent to the at least one AAI-1-peptide-solid support complex; iv) measuring the binding of the at least one AAI-1-specific labeling reagent to the at least one AAI-1-peptide-solid support complex, thereby determining at least one AAI-1-peptide binding value; v) contacting the at least one AAI-2-peptide-solid support complex with at least one AAI-2-specific labeling reagent under conditions sufficient to permit binding of the at least one AAI-2 specific labeling reagent to the at least one AAI-2-peptide-solid support complex; vi) measuring the binding of the at least one AAI-2-specific labeling reagent to the at least one AAI-2-peptide-solid support complex, thereby determining at least one AAI-2-peptide binding value; wherein: when the at least one AAI-1 peptide binding value and the at least one AAI-2 peptide binding value are combined to generate a combined peptide binding value and the combined peptide binding value is greater than a first threshold value, the subject has a cumulative reactive dose of less than a first amount of a peanut protein, and the subject is designated in Level 1; when the at least one AAI-1 peptide binding value and the at least one AAI-2 peptide binding value are combined to generate a combined peptide binding value and the combined peptide binding value is less than or equal to the first threshold value but greater than a second threshold value, the subject has a cumulative reactive dose of greater than or equal to about the first amount of a peanut protein to less than a second amount of a peanut protein, and the subject is designated in Level 2; and when the at least one AAI-1 peptide binding value and the at least one AAI-2 peptide binding value are combined to generate a combined peptide binding value and the combined peptide binding value is less than or equal to a second threshold value, the subject has a cumulative reactive dose of greater than or equal to the second amount of a peanut protein, and the subject is designated in Level 3; and b) determining the risk of anaphylaxis of the subject, wherein: for a subject in Level 1: i) the subject has a 2% risk of a Cofar grade 3 or higher reaction vs an 8% risk of a Cofar grade 2 or lower reaction after consuming ≤4 mg (CRD) of peanut; ii) the subject has a 4% risk of a Cofar grade 3 or higher reaction vs a 23% risk of a Cofar grade 2 or lower reaction after consuming ≤14 mg (CRD) of peanut; iii) the subject has a 6% risk of a Cofar grade 3 or higher reaction vs a 44% risk of a Cofar grade 2 or lower reaction after consuming ≤44 mg (CRD) of peanut; iv) the subject has an 8% risk of a Cofar grade 3 or higher reaction vs a 60% risk of a Cofar grade 2 or lower reaction after consuming ≤144 mg (CRD) of peanut; v) the subject has a 15% risk of a Cofar grade 3 or higher reaction vs a 73% risk of a Cofar grade 2 or lower reaction after consuming ≤444 mg (CRD) of peanut; vi) the subject has a 17% risk of a Cofar grade 3 or higher reaction vs an 83% risk of a Cofar grade 2 or lower reaction after consuming ≤1444 mg (CRD) of peanut; and vii) the subject has a 17% risk of a Cofar grade 3 or higher reaction vs an 83% risk of a Cofar grade 2 or lower reaction after consuming ≤4444 mg (CRD) of peanut; for a subject in Level 2: i) the subject has a 2% risk of a Cofar grade 3 or higher reaction vs a 3% risk of a Cofar grade 2 or lower reaction after consuming ≤4 mg (CRD) of peanut; ii) the subject has a 2% risk of a Cofar grade 3 or higher reaction vs an 8% risk of a Cofar grade 2 or lower reaction after consuming ≤14 mg (CRD) of peanut; iii) the subject has a 2% risk of a Cofar grade 3 or higher reaction vs a 16% risk of a Cofar grade 2 or lower reaction after consuming ≤44 mg (CRD) of peanut; iv) the subject has a 7% risk of a Cofar grade 3 or higher reaction vs a 30% risk of a Cofar grade 2 or lower reaction after consuming ≤144 mg (CRD) of peanut; v) the subject has a 10% risk of a Cofar grade 3 or higher reaction vs a 59% risk of a Cofar grade 2 or lower reaction after consuming ≤444 mg (CRD) of peanut; vi) the subject has a 13% risk of a Cofar grade 3 or higher reaction vs a 72% risk of a Cofar grade 2 or lower reaction after consuming ≤1444 mg (CRD) of peanut; and vii) the subject has a 15% risk of a Cofar grade 3 or higher reaction vs an 85% risk of a Cofar grade 2 or lower reaction after consuming ≤4444 mg (CRD) of peanut; and for a subject in Level 3: i) the subject has a 0% risk of a Cofar grade 3 or higher reaction vs a 4% risk of a Cofar grade 2 or lower reaction after consuming ≤4 mg (CRD) of peanut; ii) the subject has a 0% risk of a Cofar grade 3 or higher reaction vs a 17% risk of a Cofar grade 2 or lower reaction after consuming ≤14 mg (CRD) of peanut; iii) the subject has a 0% risk of a Cofar grade 3 or higher reaction vs a 17% risk of a Cofar grade 2 or lower reaction after consuming ≤44 mg (CRD) of peanut; iv) the subject has a 0% risk of a Cofar grade 3 or higher reaction vs a 25% risk of a Cofar grade 2 or lower reaction after consuming ≤144 mg (CRD) of peanut; v) the subject has a 4% risk of a Cofar grade 3 or higher reaction vs a 33% risk of a Cofar grade 2 or lower reaction after consuming ≤444 mg (CRD) of peanut; vi) the subject has a 13% risk of a Cofar grade 3 or higher reaction vs an 83% risk of a Cofar grade 2 or lower reaction after consuming ≤1444 mg (CRD) of peanut; and vii) the subject has a 13% risk of a Cofar grade 3 or higher reaction vs an 87% risk of a Cofar grade 2 or lower reaction after consuming ≤4444 mg (CRD) of peanut.

### BRIEF DESCRIPTION OF THE DRAWINGS

FIG. 1 shows a study schematic including DBPCFC dosing and participant information. Panel A illustrates relationships between individual doses and cumulative doses and how eliciting/reactive doses and tolerated doses are defined. Panel B shows dosing protocols used in the CAFETERIA, CoFAR6 and PEPITES studies; each follow the same dose progression protocols but differ in the initial dose with CAFETERIA starting at 3 mg and CoFAR6 and PEPITES starting at 1 mg. The dose escalation follows the PRACTALL guidelines with the exception of the 600 mg dose. Panel C shows how many subjects in each cohort reacted at each of the cumulative doses tested. To unify values seen in the CAFETERIA samples, which omitted the initial 1 mg dose, CRDs were aligned with the closest PRACTALL CRD.

FIG. 2 shows that patients reacting at lower CRDs generally had a greater number of epitopes recognized by IgE antibodies. The ordinate is the number of IgE epitopes and the abscissa is the natural logarithm of the CRD in mg of peanut protein.

FIG. 3 shows validation of an algorithm to predict CRDs. Panel A shows that the predicted score increases incrementally with the increase in CRD. The values on the ordinate represent the predicted values while those on the abscissa represent the CRD in mg of peanut protein. Panel B shows amino acid sequences and positions on conformational protein structures for the IgE-binding epitopes ara h 2_008 and ara h 3_100.

FIG. 4 shows the distribution of CRD values and the output of the predictive algorithm on samples from the CAFETERIA, CoFAR6 and PEPITES cohorts. Panel A shows that CRDs generally increase 3-fold at each of the escalation doses and the sample size at each dose tends to be small. The y-axis represents the CRD in mg of peanut protein, width represents relative frequencies of data points and the blue and red circles represent the mean and median CRD values respectively. Panel B shows that subjects in the least sensitive group (Level 3; right-hand chart) were on average 3.4 times less likely to react to a specific cumulative dose compared to those of the most sensitive group (Level 1; left-hand chart). The middle chart shows data for patients designated Level 2. For each chart, the ordinate represents the CRD in mg of peanut protein and the abscissa represents the probability of an allergic reaction in percent. Panel C shows the same data for Bins before they have been combined into Levels; Bins 1 and 2 combine to create Level 1, Bin 3 maps to Level 2 and Bin 4 to Level 3.

FIG. 5 shows probabilities of a subject from the CAFETERIA, Cofar6 and PEPITES cohorts tolerating a given cumulative dose of peanut protein based on the subject's sensitivity level. From left to right, the charts show data for patients in Levels 1 to 3, respectively, while the probabilities of tolerating a given cumulative dose in mg of peanut protein are on the ordinate.

FIG. 6 shows a representative severe reaction risk assessment by level.

### DESCRIPTION OF EMBODIMENTS

The terminology used herein is for the purpose of describing particular embodiments only and is not intended to be limiting.

Before describing several exemplary embodiments, it is to be understood that the embodiments are not limited to the details of construction or process steps set forth in the following description. The embodiments described herein are capable of modifications and of being practiced or being carried out in various ways.

Reference throughout the present disclosure to “some embodiments,” or derivations thereof, means that a particular feature, structure, material, or characteristic described in connection with the embodiment is included in at least one embodiment. Thus, the appearances of the phrases such as “in some embodiments,” in various places throughout the present disclosure is not necessarily referring to the same embodiment but can generally be attributed to any other embodiment. Furthermore, the particular features, structures, materials, or characteristics may be combined in any suitable manner in at least one embodiment.

As used herein, the terms “allergy associated immunoglobulin” and “AAI” refer to immunoglobulins in sera or plasma that mediate hypersensitivity to peanut allergens. These immunoglobulins include at least one of at least one IgE, at least one IgD, at least one IgA, at least one IgM, and at least one IgG. In some embodiments, the at least one IgG comprised at least one IgG4.

As used herein, the terms “reactive”, “reactivity”, “recognize” and the like refer to the ability of an allergy associated immunoglobulin to bind to an allergenic epitope containing peptide. The level of reactivity indicates the concentration of AAI in the serum or plasma, with high reactivity associated with higher AAI concentrations and lower reactivity associated with lower AAI concentrations. The relative AAI concentration (i.e., the relative serum or plasma reactivity) is determined by the amount of signal detected in an assay. The level of reactivity of AAI to allergenic epitope-containing peptides also indicates the intensity of the allergic response (i.e., a higher reactivity is associated with a more intense allergic reaction).

Where at least one AAI is disclosed, at least one first allergy associated immunoglobulin (AAI-1) and at least one allergy associated immunoglobulin (AAI-2) is disclosed. Where at least one AAI-peptide-solid support complex is disclosed, at least one AAI-1-peptide-solid support complex and at least one AAI-2-peptide-solid support complex is disclosed. Where at least one AAI-specific labeling reagent is disclosed, at least one AAI-1-specific labeling reagent and at least one AAI-2-specific labeling reagent is disclosed. Where at least one AAI-peptide value is disclosed, at least one AAI-1-peptide value and at least one AAI-2-peptide value is disclosed.

As used herein, the term “about” means that the recited numerical value is approximate and small variations would not significantly affect the practice of the disclosed embodiments. Where a numerical value is used, unless indicated otherwise by the context, the term “about” means the numerical value can vary by +10% and remain within the scope of the disclosed embodiments.

Where “at least one” composition of matter is disclosed, one composition of matter is disclosed. Notwithstanding the foregoing, disclosure of “at least one” composition of matter discloses n compositions of matter where n is any positive integer greater than one.

The present disclosure provides at least one first peanut peptide and at least one second peanut peptide. In some embodiments, the at least one first peanut peptide comprises the amino acid sequence WELQGDRRCQSQLER (ara h2 2.008; SEQ ID NO:1). In some embodiments, the at least one first peanut peptide comprises the amino acid sequence according to SEQ ID NO:1 but having one to four conservative amino acid substitutions therein. In some embodiments, the at least one first peanut peptide comprises the amino acid sequence according to SEQ ID NO:1 but having one conservative amino acid substitution therein. In some embodiments, the at least one first peanut peptide comprises the amino acid sequence according to SEQ ID NO: but having two conservative amino acid substitutions therein. In some embodiments, the at least one first peanut peptide comprises the amino acid sequence according to SEQ ID NO:1 but having three conservative amino acid substitutions therein. In some embodiments, the at least one first peanut peptide comprises the amino acid sequence according to SEQ ID NO:1, but having four conservative amino acid substitutions therein.

In some embodiments, the at least one second peanut peptide in the composition comprises the amino acid sequence EYDEDEYEYDEEDRR (ara h3 100; SEQ ID NO:2). In some embodiments, the at least one second peanut peptide comprises the amino acid sequence according to SEQ ID NO:2 but having one to four conservative amino acid substitutions therein. In some embodiments, the at least one second peanut peptide comprises the amino acid sequence according to SEQ ID NO:2 but having one conservative amino acid substitution therein. In some embodiments, the at least one second peanut peptide comprises the amino acid sequence according to SEQ ID NO:2 but having two conservative amino acid substitutions therein. In some embodiments, the at least one second peanut peptide comprises the amino acid sequence according to SEQ ID NO:2 but having three conservative amino acid substitutions therein. In some embodiments, the at least one second peanut peptide comprises the amino acid sequence according to SEQ ID NO:2 but having four conservative amino acid substitutions therein.

Conservative amino acid substitutions are most often classified on the basis of the amino acid structure and the general chemical characteristics of their side chains (R groups). For example, aliphatic amino acids include glycine, alanine, valine, leucine, and isoleucine, and each of these amino acids can be conservatively substituted for one another. Hydroxyl or sulfur/selenium-containing amino acids include serine, cysteine, selenocysteine, threonine, and methionine, and each of these amino acids can be conservatively substituted for one another. Aromatic amino acids include phenylalanine, tyrosine, and tryptophan, and each of these amino acids can be conservatively substituted for one another. Basic amino acids include histidine, lysine, and arginine, and each of these amino acids can be conservatively substituted for one another. Acidic or amide-containing amino acids include aspartate, glutamate, asparagine, and glutamine, and each of these amino acids can be conservatively substituted for one another.

In some embodiments, the at least one first peanut peptide is coupled to at least one solid support. In some embodiments, the at least one solid support is at least one microsphere bead, at least one glass array, at least one silicone array, at least one membrane, or at least one microtiter plate. In some embodiments, the at least one solid support is at least one glass array. In some embodiments, the at least one solid support is at least one silicone array. In some embodiments, the at least one solid support is at least one membrane. In some embodiments, the at least one solid support is at least one microtiter plate. In some embodiments, the at least one solid support is at least one microsphere bead. In some embodiments, the at least one microsphere bead is at least one avidin-coupled microsphere bead. In some embodiments, the at least one avidin-coupled microsphere bead is at least one Luminex bead such as at least one MAG™ Avidin microsphere bead or at least one LUMAVIDIN® microsphere bead. In some embodiments, at least one of the at least one solid support is coupled to at least one peanut peptide.

In some embodiments, the at least one peanut peptide is coupled to the at least one solid support by at least one linker-spacer. In some embodiments, the at least one linker-spacer comprises at least one linker chosen from at least one biotin, at least one thiol, at least one hydrazine, and at least one amine. In some embodiments, the at least one linker is at least one biotin. In some embodiments, the at least one linker is at least one thiol. In some embodiments, the at least one linker is at least one hydrazine. In some embodiments, the at least one linker is at least one amine. In some embodiments, the at least one linker-spacer comprises at least one linker-spacer chosen from at least one polypeptide, at least one oligonucleotide, at least one alkyl group, and at least one polyethylene glycol (PEG) group.

In some embodiments, the at least one spacer is at least one polypeptide. In some embodiments, the at least one spacer is at least one oligonucleotide. In some embodiments, the at least one spacer is at least one alkyl group. In some embodiments, the at least one alkyl group is at least one C₁-C₁₈alkyl group or at least one C₃-C₁₂alkyl group. In some embodiments, the at least one spacer is at least one PEG group. In some embodiments, the at least one PEG group is at least one PEG1 to PEG18. In some embodiments, the at least one PEG group is PEG12, In some embodiments, the at least one spacer is at least one alkyl group or at least one PEG group. In some embodiments, the C-terminus of at least one of the at least one peanut peptide is coupled to the at least one solid support by the at least one linker-spacer. In some embodiments, the N-terminus of at least one of the at least one peanut peptide is coupled to the at least one solid support by the at least one linker-spacer. In some embodiments, the C-terminus of at least one of the at least one peanut peptide is coupled to the at least one solid support by at least one biotin-PEG12 linker-spacer.

In some embodiments, the at least one peanut peptide can be coupled to the at least one solid support, which the at least one peanut peptide comprises a portion of a pair of click chemistry linkers and the at least one solid support comprises the remaining portion of the pair of click chemistry linkers. For example, one of the at least one peanut peptide can have one click chemistry linker, while the at least one solid support can have the remaining portion of the click chemistry linker. Examples of click chemistry linker pairs include, but are not limited to, azide-DBCO, amine-NHS ester, and thiol-malamide.

The present disclosure provides methods for determining a threshold cumulative reactive dose of a peanut peptide for a subject. The methods comprise contacting at least one first peanut peptide coupled to at least one first solid support with at least one first biological sample obtained from the subject, wherein the contacting occurs under conditions sufficient to permit binding of at least one first allergy associated immunoglobulin (AAI-1) present in the at one first biological sample to the at least one first peanut peptide to form at least one AAI-1-peptide-solid support complex. The methods also comprise contacting at least one second peanut peptide coupled to at least one second solid support with at least one second biological sample obtained from the subject, wherein the contacting is under conditions sufficient to permit binding of at least one second allergy associated immunoglobulin (AAI-2) present in the at least one second biological sample to the at least one second peanut peptide to form at least one AAI-2-peptide-solid support complex. The methods also comprise contacting the at least one AAI-1-peptide-solid support complex with at least one AAI-1-specific labeling reagent under conditions sufficient to permit binding of the at least one AAI-1 specific labeling reagent to the at least one AAI-1-peptide-solid support complex. The methods also comprise measuring the binding of the at least one AAI-1-specific labeling reagent to the at least one AAI-1-peptide-solid support complex, thereby determining at least one AAI-1-peptide binding value. The methods also comprise contacting the at least one AAI-2-peptide-solid support complex with at least one AAI-2-specific labeling reagent under conditions sufficient to permit binding of the at least one AAI-2 specific labeling reagent to the at least one AAI-2-peptide-solid support complex; and measuring the binding of the at least one AAI-2-specific labeling reagent to the at least one AAI-2-peptide-solid support complex, thereby determining at least one AAI-2-peptide binding value. When the at least one AAI-1 binding value and the at least one AAI-2 binding value are combined to generate a combined binding value and the combined binding value is greater than a first threshold value, the subject has a cumulative reactive dose of less than a first amount of a peanut protein. When the at least one AAI-1 binding value and the at least one AAI-2 binding value are combined to generate a combined binding value is less than or equal to the first threshold value but greater than a second threshold value, the subject has a cumulative reactive dose of greater than or equal to about the first amount of a peanut protein to less than a second amount of a peanut protein. When the at least one AAI-1 binding value and the at least one AAI-2 binding value are combined to generate a combined binding value and the combined binding value is less than or equal to a second threshold value, the subject has a cumulative reactive dose of greater than or equal to the second amount of a peanut protein. The steps described herein comprise an assay for detecting the presence of at least one AAI specific for at least one peanut peptide in a biological sample.

The present disclosure also provides methods for determining a threshold cumulative reactive dose of a peanut peptide for a subject. The methods comprise contacting a first peanut specific IgE as a first allergy associated immunoglobulin (AAI-1) from the subject with an AAI-1-specific labeling reagent under conditions sufficient to permit binding of the AAI-1 specific labeling reagent to the AAI-1. The methods also comprise contacting a second peanut specific IgE as a second allergy associated immunoglobulin (AAI-2) from the subject with an AAI-2-specific labeling reagent under conditions sufficient to permit binding of the AAI-2 specific labeling reagent to the AAI-2. The methods also comprise measuring the binding of the AAI-1-specific labeling reagent to the AAI-1, thereby determining an AAI-1 binding value. The methods also comprise measuring the binding of the AAI-2-specific labeling reagent to the AAI-2, thereby determining an AAI-2-peptide binding value. When the AAI-1 binding value and the AAI-2 binding value are combined to generate a combined binding value, and the combined binding value is greater than a first threshold value, the subject has a cumulative reactive dose of less than a first amount of a peanut protein. When the AAI-1 binding value and the AAI-2 binding value are combined to generate a combined binding value, and the combined binding value is less than or equal to the first threshold value but greater than a second threshold value, the subject has a cumulative reactive dose of greater than or equal to about the first amount of a peanut protein to less than a second amount of a peanut protein. When the AAI-1 binding value and the AAI-2 binding value are combined to generate a combined binding value, and the combined binding value is less than or equal to a second threshold value, the subject has a cumulative reactive dose of greater than or equal to the second amount of a peanut protein.

The present disclosure also provides methods for determining a threshold cumulative reactive dose of a peanut peptide for a subject. The methods comprise measuring the subject's allergy associated immunoglobulin activation threshold (AAI-AT) for an allergy associated immunoglobulin (AAI) directed to a peanut peptide in an effector cell activation test, thereby determining at least one AAI-peptide binding value (AAI-AT binding value). When the AAI-1-AT binding value is greater than a first threshold value, the subject has a cumulative reactive dose of less than a first amount of a peanut protein. When the AAI-AT binding value is less than or equal to the first threshold value but greater than a second threshold value, the subject has a cumulative reactive dose of greater than or equal to about the first amount of a peanut protein to less than a second amount of a peanut protein. When the AAI-AT binding value is less than or equal to a second threshold value, the subject has a cumulative reactive dose of greater than or equal to the second amount of a peanut protein. In some embodiments, the effector cell activation test comprises a basophil activation test or a mast cell activation test, or other immune cell activation test.

In any of the embodiments described herein, the methods can comprise a combination of any AAI-1 with any AAI-2 from peanut specific IgE, an IgE directed to any peanut allergen protein or any peptide derived therefrom, or Effector Cell activity thresholds (AT). Thus, any of the methods described herein and be combined whole, or in part, with the methods of any other methods.

Any of the at least one first peanut peptides and at least one second peanut peptides can be used in any of the methods described herein. In some embodiments, the first peanut peptide comprises the amino acid sequence WELQGDRRCQSQLER (ara h2 2.008; SEQ ID NO:1). In some embodiments, the at least one first peanut peptide comprises the amino acid sequence according to SEQ ID NO:1 but having one to four conservative amino acid substitutions therein. In some embodiments, the at least one first peanut peptide comprises the amino acid sequence according to SEQ ID NO:1 but having one conservative amino acid substitution therein. In some embodiments, the at least one first peanut peptide comprises the amino acid sequence according to SEQ ID NO: but having two conservative amino acid substitutions therein. In some embodiments, the at least one first peanut peptide comprises the amino acid sequence according to SEQ ID NO:1 but having three conservative amino acid substitutions therein. In some embodiments, the at least one first peanut peptide comprises the amino acid sequence according to SEQ ID NO:1, but having four conservative amino acid substitutions therein. In some embodiments, the at least one second peanut peptide in the composition comprises the amino acid sequence EYDEDEYEYDEEDRR (ara h3 100; SEQ ID NO:2). In some embodiments, the at least one second peanut peptide comprises the amino acid sequence according to SEQ ID NO:2 but having one to four conservative amino acid substitutions therein. In some embodiments, the at least one second peanut peptide comprises the amino acid sequence according to SEQ ID NO:2 but having one conservative amino acid substitution therein. In some embodiments, the at least one second peanut peptide comprises the amino acid sequence according to SEQ ID NO:2 but having two conservative amino acid substitutions therein. In some embodiments, the at least one second peanut peptide comprises the amino acid sequence according to SEQ ID NO:2 but having three conservative amino acid substitutions therein. In some embodiments, the at least one second peanut peptide comprises the amino acid sequence according to SEQ ID NO:2 but having four conservative amino acid substitutions therein.

In some embodiments, the at least one first peanut peptide is coupled to at least one first microsphere bead. In some embodiments, the at least one second peanut peptide is coupled to at least one second microsphere bead. In some embodiments, the at least one first microsphere bead and the at least one second microsphere bead are different microsphere beads. In some embodiments, the at least one first microsphere bead and the at least one second microsphere bead are the same microsphere bead. At least one of the at least one first peanut peptide and the at least one second peanut peptide can be coupled to at least one solid support by any of the linker-spacers described herein. At least one of the at least one first peanut peptide and the at least one second peanut peptide can be coupled to at least one solid support by at least one of the C-terminal or N-terminal end as described herein.

The at least one biological sample can be any biological sample obtained from a subject. In some embodiments, the at least one biological sample is chosen from serum, plasma, saliva, or at least one buccal swab. In some embodiments, the at least one biological sample is serum or plasma. In some embodiments, the at least one biological sample is serum. In some embodiments, the at least one biological sample is plasma. In some embodiments, the at least one biological sample is saliva. In some embodiments, the at least one biological sample is at least one buccal swab.

The AAIs that may be present in the at least one biological sample from a subject may include at least one of at least one IgM, at least one IgA, at least one IgD, at least one IgG, or at least one IgE. In some embodiments, the AAI in the at least one biological sample is at least one IgM, at least one IgA, or at least one IgD. In some embodiments, the AAI in the at least one biological sample is at least one IgG or at least one IgE. In some embodiments, the AAI in the at least one biological sample is at least one IgE. In some embodiments, the AAI in the at least one biological sample is at least one IgG. In some embodiments, the at least one IgG is at least one IgG4.

In some embodiments, the at least one AAI-specific labeling reagent is at least one detectably labeled anti-human antibody. In some embodiments, the at least one detectably labeled anti-human antibody is at least one detectably labeled anti-human IgA antibody. In some embodiments, the at least one detectably labeled anti-human antibody is at least one detectably labeled anti-human IgD antibody. In some embodiments, the at least one detectably labeled anti-human antibody is at least one detectably labeled anti-human IgM antibody. In some embodiments, the at least one detectably labeled anti-human antibody is at least one detectably labeled anti-human IgG antibody. In some embodiments, the at least one detectably labeled anti-human IgG antibody is at least one detectably labeled anti-human IgG4 antibody. In some embodiments, the at least one detectably labeled anti-human antibody is at least one detectably labeled anti-human IgE antibody.

In some embodiments, the at least one detectable label of the at least one AAI-specific labeling reagent comprises at least one fluorochrome chosen from phycoerythrin (PE), at least one cyanine dye, at least one fluorescent dye, at least one infrared (IR) dye, or at least one fluorescent protein. In some embodiments, the detectable label of the AAI-specific labeling reagent comprises at least one chromogenic dye, at least one enzyme label, or at least one radioactive label. In some embodiments, the at least one detectable label of the at least one AAI-specific labeling reagent is PE. In some embodiments, the at least one detectable label of the at least one AAI-specific labeling reagent is at least one cyanine dye. In some embodiments, the at least one cyanine dye is Cy3 or Cy5. In some embodiments, the at least one detectable label of the at least one AAI-specific labeling reagent is at least one fluorescent dye. In some embodiments, the at least one fluorescent dye is Texas Red or Alexa-fluor. In some embodiments, the at least one detectable label of the at least one AAI-specific labeling reagent is at least one IR dye. In some embodiments, the at least one detectable label of the at least one AAI-specific labeling reagent is at least one fluorescent protein. In some embodiments, the at least one detectable label of the at least one AAI-specific labeling reagent is at least one chromogenic dye. In some embodiments, the at least one detectable label of the at least one AAI-specific labeling reagent is at least one enzyme label. In some embodiments, the at least one detectable label of the at least one AAI-specific labeling reagent is at least one radioactive label. In some embodiments, the at least one enzyme label is at least one horse radish peroxidase (HRP) or at least one alkaline phosphatase. In some embodiments, the at least one detectable label of the at least one AAI-specific labeling reagent is at least one HRP. In some embodiments, the at least one detectable label of the at least one AAI-specific labeling reagent is at least one alkaline phosphatase. In some embodiments, the at least one AAI-specific labeling reagent is at least one PE-labeled anti-human IgE antibody. In some embodiments, a single detectable label can generally be used for universal detection of all complexes.

In some embodiments, the at least one anti-human AAI antibody may be conjugated to at least one reporter moiety that is not directly detectable, so that specific binding of at least one second, directly detectable reporter moiety to the at least one AAI-specific labeling reagent is necessary for analysis of binding. For example, at least one biotin-conjugated anti-AAI antibody can be used in combination with at least one streptavidin-conjugated fluorescent dye for detection of the at least one biotin-conjugated anti-AAI. Examples of indirectly-detectable reporter moieties include biotin, digoxigenin, and other haptens that are detectable upon subsequent binding of at least one secondary antibody (e.g., anti-digoxigenin) or other binding partner (e.g., streptavidin) which is labeled for direct detection.

In some embodiments, the measuring of the binding of the at least one AAI-specific labeling reagent to at least one AAI-peptide-solid support complex is carried out by at least one point of care device. In some embodiments, the at least one point of care device is at least one multiplex peptide-bead flow cytometric analysis device or at least one lateral flow assay device. In some embodiments, the at least one detectable label can be observed via silver staining, quantum dots, or refraction methodologies.

Any of the foregoing embodiments may be in the form of at least one microarray immunoassay, wherein the at least one first peanut peptide is bound to one well of at least one microtiter plate and the at least one second peanut peptide is bound to another well of the at least one microtiter plate. Thereafter, the at least one first peanut peptide and the at least one second peanut peptide are reacted with at least one biological sample to bind AAI. The at least one first peanut peptide and the at least one second peanut peptide may also be used in at least one lateral flow immunoassay format, wherein the at least one first peanut peptide and the at least one second peanut peptide are immobilized in at least one discrete area on at least one porous support or at least one chromatographic support, and the serum or plasma of the at least one biological sample is wicked through the at least one porous support or the at least one chromatographic support to contact the at least one first peanut peptide and the at least one second peanut peptide for binding of AAI to the at least one first peptide and the at least one second peptide. In this assay, the at least one AAI-specific labeling reagent may comprise at least one chromophore or at least one dye conjugated to the at least one anti-AAI antibody. The at least one AAI-specific labeling reagent is also wicked through the at least one porous support or the at least one chromatographic support to contact the at least one peptide-AAI complex for binding of the at least one AAI-specific labeling reagent to the at least one peptide-AAI complex, which indicates the presence or absence in the serum or plasma of at least one antibody to the at least one first peanut peptide or the at least one second peanut peptide immobilized at each discrete location of the at least one porous or chromatographic support.

Any of the foregoing embodiments may also be in the form of at least one flow cytometry assay in which at least one of the at least one first peanut peptide and the at least one second peanut peptide is coupled to at least one separately identifiable solid support suitable for analysis by flow cytometry, such as at least one bead. In some embodiments, the at least one bead with at least one of the at least one first coupled peptide and the at least one second coupled peptide is contacted with the at least one biological sample of a subject to bind any peptide-specific AAI that is bound to the at least one bead via at least one of the at least one first peanut peptide and the at least one second peanut peptide, thus forming at least one of at least one first peptide-AAI complex and at least one second peptide-AAI complex on the at least bead. At least one AAI-specific labeling reagent comprising, for example, at least one fluorescent reporter moiety, is then bound to the at least one peptide-AAI complex and the at least one bead is analyzed quantitatively or qualitatively by flow cytometry. This technique detects fluorescence from the bound at least one AAI-specific labeling reagent associated with the at least one bead to which at least one of the at least one first peanut peptide and the at least one second peanut peptide is coupled.

In some embodiments, the at least one flow cytometry assay may be at least one multiplex assay, such as provided by LUMINEX®, which uses at least one microsphere array platform for quantitation and detection of at least one peptide and at least one protein. At least one of the at least one first peanut peptide and the at least one second peanut peptide is bound to at least one set of beads with the same or different spectral properties that can be used to quantify the associated at least one first peanut peptide or at least one second peanut protein bound to AAI by flow cytometry. The at least one set of bead is then contacted with the at least one biological sample of a subject to bind peptide-recognizing AAI to the at least one bead to form at least one peptide-AAI complex on the at least one bead and at least one AAI-specific labeling reagent comprising, for example, at least one fluorescent reporter moiety bound to the AAI of the at least one peptide-AAI complex. The at least one bead is analyzed by monitoring at least one spectral property of the at least one bead and the amount of associated fluorescence from the bound at least one AAI-specific labeling reagent. This process allows quantification of at least one of the at least one peanut peptide and the at least one second peanut polypeptide on the at least one bead and the presence or absence of AAI that is reactive to the at least one of the at least one peanut peptide and the at least one second peanut polypeptide. Results of the at least one assay are interpreted as discussed herein.

A particularly useful quantitative assay for use in any of the methods described herein is at least one multiplex peptide-bead assay for flow cytometric analysis, such as the LUMINEX® exMAP multiplex bead assay, which is a high-throughput alternative to an ELISA. In this assay, at least one microsphere dyed with distinct proportions of at least one red fluorophore and at least one near-infrared fluorophore are used as at least one solid support. At least one of the at least one first peanut peptide and the at least one second peanut peptide may be chemically linked to the at least one microsphere or bound thereto through at least one peptide-specific capture antibody coated on the at least one microsphere. The proportions of the at least one red fluorophore and the at least one near-infrared fluorophore define a “spectral address” for each population comprising at least one microsphere that can be identified by at least one flow cytometer using digital signal processing. Detection of a third fluorescence color is used for measurement of the fluorescence intensity of the at least one reporter moiety of the at least one AAI-specific labeling reagent bound to the at least one microsphere. Multiple analytes can be detected simultaneously by binding at least one of the at least one first peanut peptide or at least one second peanut peptide to at least one microsphere having a specific “spectral address.” Contacting the at least one microsphere with at least one biological sample containing AAI that is or are specific for the at least one first peanut peptide or the at least one second peanut peptide bound to the at least one microsphere is followed by addition of at least one anti-human AAI antibody conjugated to at least one reporter moiety. In some embodiments, the at least one reporter moiety of the at least one anti-human AAI antibody is biotin and binding to phycoerythyrin (PE)-conjugated streptavidin provides at least one fluorescent signal for detection. Following binding of the at least one AAI-specific labeling reagent, the at least one microsphere is analyzed on at least one dual-laser flow-based detection instrument, such as the LUMINEX® 200 or BIO-RAD® BIO-PLEX® analyzer. At least one first laser classifies the at least one microsphere and identifies the at least one first peanut peptide or the at least one second peanut peptide bound to the at least one microsphere. The at least one second laser determines the magnitude of the at least one reporter-derived signal, which is in direct proportion to the amount of bound serum or plasma AAI. In some embodiments, the at least one microsphere comprises at least one polystyrene bead.

In some embodiments, the methods comprise contacting at least one AAI-1-peptide-solid support complex with at least one AAI-1-specific labeling reagent under conditions sufficient to permit binding of the at least one AAI-1 specific labeling reagent to the at least one AAI-1-peptide-solid support complex, thereby determining at least one AAI-1-peptide binding value. In some embodiments, the methods comprise contacting at least one AAI-2-peptide-solid support complex with at least one AAI-2-specific labeling reagent under conditions sufficient to permit binding of the at least one AAI-2 specific labeling reagent to the at least one AAI-2-peptide-solid support complex, thereby determining at least one AAI-1-peptide binding value. In some embodiments, measuring the binding of the at least one AAI-specific labeling reagent to at least one AAI-peptide-solid support complex comprises measuring the mean fluorescent intensity or median fluorescent intensity (MFI) of the at least AAI-specific labeling reagent. In some embodiments, at least one of the at least one AAI-1-peptide binding value and the at least one AAI-2-peptide binding value comprises a mean fluorescent intensity. In some embodiments, at least one of the at least one AAI-1-peptide binding value and the at least one AAI-2-peptide binding value comprises an MFI. When the at least one AAI-1 binding value and the at least one AAI-2 binding value are combined to generate a combined binding value and the combined binding value is greater than a first threshold value, the subject has a cumulative reactive dose of less than a first amount of a peanut protein. When the at least one AAI-1 binding value and the at least one AAI-2 binding value are combined to generate a combined binding value and the combined binding value is less than or equal to the first threshold value but greater than a second threshold value, the subject has a cumulative reactive dose of greater than or equal to about the first amount of a peanut protein to less than a second amount of a peanut protein. When the at least one AAI-1 binding value and the at least one AAI-2 binding value are combined to generate a combined binding value and the combined binding value is less than or equal to a second threshold value, the subject has a cumulative reactive dose of greater than or equal to the second amount of a peanut protein. In some embodiments, the AAI-specific labeling reagent is at least one of the at least one detectably labeled anti-human antibody described herein. In some embodiments, the MFI of the at least one AAI-specific labeling reagent is background subtracted.

The present disclosure also provides methods for determining the risk of a severe versus a non-severe reaction to peanuts in a subject allergic to peanuts. The methods comprise determining a threshold cumulative reactive dose of a peanut peptide for the subject. Any of such methods described herein can be employed. In some embodiments, the methods comprise: a) contacting at least one first peanut peptide comprising the amino acid sequence WELQGDRRCQSQLER (SEQ ID NO:1), or an amino acid sequence comprising SEQ ID NO:1 but having one to four conservative amino acid substitutions therein, coupled to at least one first solid support with at least one first biological sample obtained from the subject, wherein the contacting is under conditions sufficient to permit binding of at least one allergy associated immunoglobulin (AAI-1) present in the at least one first biological sample to the at least one first peanut peptide to form at least one AAI-1-peptide-solid support complex; b) contacting at least one second peanut peptide comprising the amino acid sequence EYDEDEYEYDEEDRR (SEQ ID NO:2), or an amino acid sequence comprising SEQ ID NO:2 but having one to four conservative amino acid substitutions therein, coupled to at least one second solid support with at least one second biological sample obtained from the subject, wherein the contacting is under conditions sufficient to permit binding of at least one second allergy associated immunoglobulin (AAI-2) present in the at least one second biological sample to the at least one second peanut peptide to form at least one AAI-2-peptide-solid support complex; c) contacting the at least one AAI-1-peptide-solid support complex with at least one AAI-1-specific labeling reagent under conditions sufficient to permit binding of the at least one AAI-1 specific labeling reagent to the at least one AAI-1-peptide-solid support complex; d) measuring the binding of the at least one AAI-1-specific labeling reagent to the at least one AAI-1-peptide-solid support complex, thereby determining at least one AAI-1-peptide binding value; e) contacting the at least one AAI-2-peptide-solid support complex with at least one AAI-2-specific labeling reagent under conditions sufficient to permit binding of the at least one AAI-2 specific labeling reagent to the at least one AAI-2-peptide-solid support complex; and f) measuring the binding of the at least one AAI-2-specific labeling reagent to the at least one AAI-2-peptide-solid support complex, thereby determining at least one AAI-2-peptide binding value.

In such methods, when the at least one AAI-1 peptide binding value and the at least one AAI-2 peptide binding value are combined to generate a combined peptide binding value and the combined peptide binding value is greater than a first threshold value, the subject has a cumulative reactive dose of less than a first amount of a peanut protein, and the subject is designated in Level 1; when the at least one AAI-1 peptide binding value and the at least one AAI-2 peptide binding value are combined to generate a combined peptide binding value and the combined peptide binding value is less than or equal to the first threshold value but greater than a second threshold value, the subject has a cumulative reactive dose of greater than or equal to about the first amount of a peanut protein to less than a second amount of a peanut protein, and the subject is designated in Level 2; and when the at least one AAI-1 peptide binding value and the at least one AAI-2 peptide binding value are combined to generate a combined peptide binding value and the combined peptide binding value is less than or equal to a second threshold value, the subject has a cumulative reactive dose of greater than or equal to the second amount of a peanut protein, and the subject is designated in Level 3.

The methods further comprise determining the risk of a severe versus a non-severe reaction to peanuts in a subject allergic to peanuts. In some embodiments, the severe versus non-severe reaction to peanuts is defined by the Cofar allergy severity index (Chinthrajah et al., J. Allergy. Clin. Immunol., 2022, 149, 2166-2170). Cofar Grade 1 (non-severe) is characterized by no impact on activity, no intervention is needed, and no or minimal medical intervention is required. It is characterized by a reaction involving one of the following organ systems in which the symptoms are mild: i) cutaneous: limited or localized hives, swelling (e.g., lip edema), skin flushing (e.g., few areas of faint erythema) or mild pruritus; ii) upper respiratory: rhinitis (not severe or persistent), cough unrelated to laryngeal edema or bronchospasm, throat discomfort; iii) conjunctival: injection/redness, itching, tearing; and iv) GI: local oral or pharyngeal symptoms (e.g., pruritus), nausea or abdominal pain (without persistence or change in activity level), single episode of vomiting and/or single episode of diarrhea. Cofar Grade 2 (non-severe) is characterized by a reaction involving two or more of the following organ systems in which the symptoms are mild: i) cutaneous: limited or localized hives, swelling (e.g., lip edema), skin flushing (e.g., few areas of faint erythema) or mild pruritus; ii) upper respiratory: rhinitis (not severe or persistent), cough unrelated to laryngeal edema or bronchospasm, throat discomfort; iii) conjunctival: injection/redness, itching, tearing; and iv) GI: local oropharyngeal symptoms, nausea or abdominal pain (without persistence or change in activity level), single episode of vomiting and/or single episode of diarrhea. Alternately, Cofar Grade 2 (non-severe) is characterized by a reaction involving moderate symptoms in at least one of the following organ systems: i) cutaneous (both moderate and severe cutaneous symptoms are captured as grade 2): generalized pruritus/urticaria/erythema, significant angioedema excluding lip swelling; ii) upper respiratory: severe and persistent rhinitis, persistent cough unrelated to laryngeal edema or bronchospasm, throat tightness without hoarseness; and iii) GI: persisting nausea or abdominal pain (with change in activity level), two episodes of vomiting and/or diarrhea. Cofar Grade 3 (severe) is characterized by a reaction involving one or more of the following features: i) lower respiratory: wheezing, chest tightness, dyspnea or cough; responsive to first-line treatment (e.g. 1-2 doses of IM epinephrine with or without supplemental oxygen); ii) laryngeal: throat tightness with hoarseness, odynophagia; and iii) GI: severe persisting abdominal pain, >2 episodes of vomiting and/or diarrhea. Cofar Grade 4 (severe) is characterized by life-threatening reaction involving one or more of the following features (with or without other symptoms listed in grades 1-3): i) lower respiratory: throat tightness with stridor; or wheezing, chest tightness, dyspnea, or cough associated with: a requirement for supplemental oxygen, and refractory to treatment (refractory to treatment also implied by need for IV epinephrine infusion or continuous albuterol nebulizer following 2 doses of IM epinephrine) (poor response to 2 doses of IM epinephrine); or respiratory compromise requiring mechanical support; or ii) cardiovascular: reduced blood pressure (BP) with associated symptoms of end-organ dysfunction (e.g., hypotonia (collapse) and syncope) defined as: a decrease in systolic BP greater than 30% from that person's baseline or systolic BP less than: Children ≤10 years: 70 mmHg+(2× age in years) or adults and children >10 years: ≤90 mmHg, or (as per WAO anaphylaxis guideline). Cofar Grade 5 (severe) is characterized by death.

Cofar grade 3 or higher can be considered to be a severe reaction to peanuts. Cofar grade 2 or lower can be considered to be a non-severe reaction to peanuts. In some embodiments, the severe versus non-severe reaction to peanuts is defined by requiring epinephrine (severe) versus not requiring epinephrine (non-severe). In some embodiments, the severe versus non-severe reaction to peanuts is defined by experiencing anaphylaxis (severe) versus not experiencing anaphylaxis (non-severe).

For a subject in Level 1: i) the subject has a 2% risk of a Cofar grade 3 or higher reaction vs an 8% risk of a Cofar grade 2 or lower reaction after consuming ≤4 mg (CRD) of peanut; ii) the subject has a 4% risk of a Cofar grade 3 or higher reaction vs a 23% risk of a Cofar grade 2 or lower reaction after consuming ≤14 mg (CRD) of peanut; iii) the subject has a 6% risk of a Cofar grade 3 or higher reaction vs a 44% risk of a Cofar grade 2 or lower reaction after consuming ≤44 mg (CRD) of peanut; iv) the subject has an 8% risk of a Cofar grade 3 or higher reaction vs a 60% risk of a Cofar grade 2 or lower reaction after consuming ≤144 mg (CRD) of peanut; v) the subject has a 15% risk of a Cofar grade 3 or higher reaction vs a 73% risk of a Cofar grade 2 or lower reaction after consuming ≤444 mg (CRD) of peanut; vi) the subject has a 17% risk of a Cofar grade 3 or higher reaction vs an 83% risk of a Cofar grade 2 or lower reaction after consuming ≤1444 mg (CRD) of peanut; and vii) the subject has a 17% risk of a Cofar grade 3 or higher reaction vs an 83% risk of a Cofar grade 2 or lower reaction after consuming ≤4444 mg (CRD) of peanut.

For a subject in Level 2: i) the subject has a 2% risk of a Cofar grade 3 or higher reaction vs a 3% risk of a Cofar grade 2 or lower reaction after consuming ≤4 mg (CRD) of peanut; ii) the subject has a 2% risk of a Cofar grade 3 or higher reaction vs an 8% risk of a Cofar grade 2 or lower reaction after consuming ≤14 mg (CRD) of peanut; iii) the subject has a 2% risk of a Cofar grade 3 or higher reaction vs a 16% risk of a Cofar grade 2 or lower reaction after consuming ≤44 mg (CRD) of peanut; iv) the subject has a 7% risk of a Cofar grade 3 or higher reaction vs a 30% risk of a Cofar grade 2 or lower reaction after consuming ≤144 mg (CRD) of peanut; v) the subject has a 10% risk of a Cofar grade 3 or higher reaction vs a 59% risk of a Cofar grade 2 or lower reaction after consuming ≤444 mg (CRD) of peanut; vi) the subject has a 13% risk of a Cofar grade 3 or higher reaction vs a 72% risk of a Cofar grade 2 or lower reaction after consuming ≤1444 mg (CRD) of peanut; and vii) the subject has a 15% risk of a Cofar grade 3 or higher reaction vs an 85% risk of a Cofar grade 2 or lower reaction after consuming ≤4444 mg (CRD) of peanut.

For a subject in Level 3: i) the subject has a 0% risk of a Cofar grade 3 or higher reaction vs a 4% risk of a Cofar grade 2 or lower reaction after consuming ≤4 mg (CRD) of peanut; ii) the subject has a 0% risk of a Cofar grade 3 or higher reaction vs a 17% risk of a Cofar grade 2 or lower reaction after consuming ≤14 mg (CRD) of peanut; iii) the subject has a 0% risk of a Cofar grade 3 or higher reaction vs a 17% risk of a Cofar grade 2 or lower reaction after consuming ≤44 mg (CRD) of peanut; iv) the subject has a 0% risk of a Cofar grade 3 or higher reaction vs a 25% risk of a Cofar grade 2 or lower reaction after consuming ≤144 mg (CRD) of peanut; v) the subject has a 4% risk of a Cofar grade 3 or higher reaction vs a 33% risk of a Cofar grade 2 or lower reaction after consuming ≤444 mg (CRD) of peanut; vi) the subject has a 13% risk of a Cofar grade 3 or higher reaction vs an 83% risk of a Cofar grade 2 or lower reaction after consuming ≤1444 mg (CRD) of peanut; and vii) the subject has a 13% risk of a Cofar grade 3 or higher reaction vs an 87% risk of a Cofar grade 2 or lower reaction after consuming ≤4444 mg (CRD) of peanut.

Determining the risk of a severe versus a non-severe reaction to peanuts in a subject allergic to peanuts can be carried out by generating or using a reactivity database. This is a database of patients who have undergone standardized (e.g. PRACTALL) oral food challenges (OFCs) to determine a severe versus a non-severe reaction to peanuts. Specifically, the database records at which amount of ingested peanut each patient reached a state of severe reaction, such as anaphylaxis, and also the results of the peanut threshold test for this patient. This allows the determination of the percentage of patients in each level (1, 2 or 3) reach a severe reaction, such as anaphylaxis. Any standardized OFCs database can be used and the database can be extended and made more generalizable by incorporating more patient data. Furthermore, the symptoms observed during the OFC can be graded using a standardized severity index such as Cofar. This was done for the 137 subjects used in the example herein. For example, blood can be sampled from the patient and assessed by the previously described peanut threshold test that places the patient into one of three levels of epitope reactivity to epitopes h2.008 and h3.100. Depending on the patient's level, the risk of a severe or non-severe reaction for an amount of peanut consumed can be determined (see Example 4).

Any of the at least one first peanut peptide coupled to any of the at least one first solid support described herein can be used. Similarly, any of the at least one second peanut peptide coupled to any of the at least one second solid support described herein can be used. For example, in some embodiments, at least one of the at least one first peanut peptide comprises the amino acid sequence according to SEQ ID NO:1 or an amino acid sequence comprising SEQ ID NO:1 but having one to four conservative amino acid substitutions therein, and at least one of the at least one second peanut peptide comprises the amino acid sequence according to SEQ ID NO:2 or an amino acid sequence comprising SEQ ID NO:2 but having one to four conservative amino acid substitutions therein. In some embodiments, at least one of the at least one first peanut peptide comprises the amino acid sequence according to SEQ ID NO:1 and at least one of the at least one second peanut peptide comprises the amino acid sequence according to SEQ ID NO:2.

In some embodiments, at least one of the at least one first peanut peptide and the at least one second peanut peptide is coupled to at least one microsphere bead. At least one of the at least one first peanut peptide and the at least one second peanut peptide can be coupled to at least one solid support by any of the linker-spacers described herein. At least one of the at least one first peanut peptide and the at least one second peanut peptide can be coupled to the at least one solid support by at least one C-terminal or N-terminal end as described herein.

The at least one biological sample can be any biological sample obtained from a subject. In some embodiments, the at least one biological sample is chosen from serum, plasma, saliva, or at least one buccal swab. In some embodiments, the at least one biological sample is serum or plasma. In some embodiments, the at least one biological sample is serum. In some embodiments, the at least one biological sample is plasma. In some embodiments, the at least one biological sample is saliva. In some embodiments, the at least one biological sample is at least one buccal swab.

The AAIs that may be present in the at least one biological sample from a subject may include at least one of at least one IgM, at least one IgA, at least one IgD, at least one IgG, and at least one IgE. In some embodiments, the AAI in the at least one biological sample is at least one of at least one IgM, at least one IgA, and at least one IgD. In some embodiments, the AAI in the at least one biological sample is at least one of at least one IgG and at least one IgE. In some embodiments, the AAI in the at least one biological sample is at least one IgE. In some embodiments, the AAI in the at least one biological sample is at least one IgG. In some embodiments, the at least one IgG is at least one IgG4.

In some embodiments, the at least one AAI-specific labeling reagent is at least one detectably labeled anti-human antibody. In some embodiments, the at least one detectably labeled anti-human antibody is at least one detectably labeled anti-human IgA antibody. In some embodiments, the at least one detectably labeled anti-human antibody is at least one detectably labeled anti-human IgD antibody. In some embodiments, the at least one detectably labeled anti-human antibody is at least one detectably labeled anti-human IgM antibody. In some embodiments, the at least one detectably labeled anti-human antibody is at least one detectably labeled anti-human IgG antibody. In some embodiments, the at least one detectably labeled anti-human IgG antibody is at least one detectably labeled anti-human IgG4 antibody. In some embodiments, the at least one detectably labeled anti-human antibody is at least one detectably labeled anti-human IgE antibody.

In some embodiments, the at least one detectable label of the at least one AAI-specific labeling reagent is a fluorochrome chosen from phycoerythrin (PE), at least one cyanine dye, at least one fluorescent dye, an at least one infrared dye, and a fluorescent protein. In some embodiments, the at least one detectable label of the at least one AAI-specific labeling reagent is at least one chromogenic dye, at least one enzyme label, and at least one radioactive label. In some embodiments, the at least one detectable label of the at least one AAI-specific labeling reagent is PE. In some embodiments, the at least one detectable label of the at least one AAI-specific labeling reagent is at least one cyanine dye. In some embodiments, the at least one cyanine dye is Cy3 or Cy5. In some embodiments, the at least one detectable label of the at least one AAI-specific labeling reagent is at least one fluorescent dye. In some embodiments, the at least one fluorescent dye is Texas Red or Alexa-fluor. In some embodiments, the at least one detectable label of the at least one AAI-specific labeling reagent is at least one IR dye. In some embodiments, the at least one detectable label of the at least one AAI-specific labeling reagent is at least one chromogenic dye. In some embodiments, the at least one detectable label of the at least one AAI-specific labeling reagent is at least one enzyme label. In some embodiments, the at least one detectable label of the at least one AAI-specific labeling reagent is at least one radioactive label. In some embodiments, the at least one enzyme label is horse radish peroxidase (HRP) or at least one alkaline phosphatase. In some embodiments, the at least one detectable label of the at least one AAI-specific labeling reagent is HRP. In some embodiments, the at least one detectable label of the at least one AAI-specific labeling reagent is at least one alkaline phosphatase. In some embodiments, the AAI-specific labeling reagent is at least one PE-labeled anti-human IgE antibody. In some embodiments, a single detectable label can generally be used for universal detection of all complexes.

In some embodiments, the at least one anti-human AAI antibody may be conjugated to at least one reporter moiety that is not directly detectable, so that specific binding of at least one second, directly detectable reporter moiety to the at least one AAI-specific labeling reagent is necessary for analysis of binding. For example, at least one biotin-conjugated anti-AAI antibody can be used in combination with at least one streptavidin-conjugated fluorescent dye for detection of the at least one biotin-conjugated anti-AAI. Non-limiting examples of at least one indirectly-detectable reporter moiety include biotin, digoxigenin, and at least one other haptens that is detectable upon subsequent binding of at least one secondary antibody (e.g., anti-digoxigenin) or at least one other binding partner (e.g., streptavidin) which is labeled for direct detection.

In some embodiments, the measuring of the binding of the at least one AAI-specific labeling reagent to each of the at least one AAI-peptide-solid support complex is carried out by at least one point of care device. In some embodiments, the at least one point of care device is at least one multiplex peptide-bead flow cytometric analysis device or at least one lateral flow assay device. In some embodiments, the at least one detectable label can be observed via silver staining, at least one quantum dot, or at least one refraction methodology.

Any of the foregoing embodiments may be in the form of at least one microarray immunoassay, wherein at least one of the at least one first peanut peptide and the at least one second peanut peptide is bound to at least one separate well of at least one microtiter plate and reacted with at least one biological sample to bind at least one AAJ. At least one of the at least one first peanut peptide and the at least one second peanut peptide may also be used in at least one lateral flow immunoassay format, wherein at least one of the at least one first peanut peptide and the at least one second peanut peptide is immobilized in a discrete area on at least one porous support or at least one chromatographic support and the serum or plasma is wicked through the at least one porous support or at least one chromatographic support to contact at least one of the at least one first peanut peptide or the at least one second peanut peptide for binding of the AAI to at least one of the at least one first peanut peptide and the at least one second peanut peptide. In this assay, the at least one AAI-specific labeling reagent may comprise at least one chromophore or at least one dye conjugated to at least one anti-AAI antibody. The at least one AAI-specific labeling reagent is also wicked through the at least one porous support or the at least one chromatographic support to contact the at least one peptide-AAI complex for binding of the at least one AAI-specific labeling reagent to the at least one peptide-AAI complex, which indicates the presence or absence in the serum or plasma of at least one antibody to at least one of the at least one first peanut peptide and the at least one second peanut peptide immobilized at each discrete location of the at least one porous support or the at least one chromatographic support.

Any of the foregoing embodiments may also be in the form of at least one flow cytometry assay in which at least one of the at least one first peanut peptide and the at least one second peanut peptide is coupled to at least one separately identifiable solid support suitable for analysis by flow cytometry, such as at least one bead. In some embodiments, the at least one bead coupled to at least one of the at least one first peanut peptide and the at least one second peanut peptide is contacted with the at least one biological sample of a subject to bind any peptide-specific AAI that is bound to the at least one bead via at least one of the at least one first peanut peptide and the at least one second peanut peptide, thus forming at least one peptide-AAI complex on the at least one bead. At least one AAI-specific labeling reagent comprising, for example, at least one fluorescent reporter moiety, is then bound to the at least one peptide-AAI complex and the at least one bead is analyzed quantitatively or qualitatively by flow cytometry. This technique detects fluorescence from the bound AAI-specific labeling reagent associated with the at least one bead to which at least one of the at least one first peanut peptide and the at least one second peanut peptide is coupled.

In some embodiments, the flow cytometry assay may be at least one multiplex assay, such as provided by LUMINEX®, which uses at least one microsphere array platform for quantitation and detection of peptides and proteins. At least one of the at least one first peanut peptide and the at least one second peanut peptide is bound to at least one set of beads with the same or different spectral properties that can be used to quantify at least one of the at least one first peanut peptide and the at least one second peanut peptide bound to AAI by flow cytometry. The at least one set of beads is then contacted with the at least one biological sample of a subject to bind at least one peptide-recognizing AAI to at least one bead to form at least one peptide-AAI complex on the at least one bead, and at least one AAI-specific labeling reagent comprising, by way of a non-limiting example, at least one fluorescent reporter moiety bound to the AAI of the at least one peptide-AAI complex. The at least one bead is analyzed by monitoring at least one spectral property of the at least one bead and the amount of associated fluorescence from the at least one bound labeling reagent. This process allows quantification of the at least one of the at least one first peanut peptide and the at least one second peanut peptide on the at least one bead and the presence or absence of AAI that is reactive to the at least one of the at least one first peanut peptide and the at least one second peanut peptide. Results of the assay are interpreted as discussed herein.

A particularly useful quantitative assay for use in any of the methods described herein is at least one multiplex peptide-bead assay for flow cytometric analysis, such as the LUMINEX® exMAP multiplex bead assay, which is a high-throughput alternative to an ELISA. In this assay, at least one microsphere dyed with distinct proportions of at least one red fluorophore and at least one near-infrared fluorophore are used as the at least one solid support. At least one of the at least one first peanut peptide and the at least one second peanut peptide may be chemically linked to the at least one microsphere or bound thereto through at least one peptide-specific capture antibodies coated on the at least one microsphere. The proportions of the at least one fluorophore define a “spectral address” for the at least one microsphere population that can be identified by at least one flow cytometer using digital signal processing. Detection of a third fluorescence color is used for measurement of the fluorescence intensity of the at least one reporter moiety of the at least one AAI-specific labeling reagent bound to the at least one microsphere. Multiple analytes can be detected simultaneously by binding at least one of at least one first peanut peptide and at least one second peanut peptide to at least one microsphere having at least one specific “spectral address.” Contacting the at least one microsphere with at least one biological sample containing AAI that is specific for at least one of the at least one first peanut peptide and the at least one second peanut bound to the AAI is followed by addition of at least one anti-human AAI antibody conjugated to at least one reporter moiety. In some embodiments, the at least one reporter moiety of the at least one anti-human AAI antibody is biotin and binding to phycoerythyrin (PE)-conjugated streptavidin provides the fluorescent signal for detection. Following binding of the at least one AAI-specific labeling reagent, the at least one microsphere is analyzed on at least one dual-laser flow-based detection instrument, such as the LUMINEX® 200 or BIO-RAD® BIO-PLEX® analyzer. One laser classifies the at least one microsphere and identifies at least one of the at least one first peanut peptide and the at least one second peanut peptide bound to the at least one microsphere. The second laser determines the magnitude of the at least one reporter-derived signal, which is in direct proportion to the amount of bound serum AAI or bound plasma AAI. In some embodiments, the at least one microsphere comprises at least one polystyrene bead.

In some embodiments, measuring the binding of the at least one AAI-specific labeling reagent to the at least one AAI-peptide-solid support complex comprises measuring the MFI of the at least one AAI-specific labeling reagent as described herein. The methods also comprise measuring the binding of at least one AAI-1-specific labeling reagent to at least one AAI-1-peptide-solid support complex, thereby determining at least one AAI-1-peptide binding value. The methods also comprise measuring the binding of the at least one AAI-2-specific labeling reagent to the at least one AAI-2-peptide-solid support complex, thereby determining at least one AAI-2-peptide binding value. The methods also comprise combining the at least one AAI-1 binding value and the at least one AAI-2 binding value to generate a combined binding value. The methods also comprise combining at least third AAI-1 binding value and the at least fourth AAI-2 binding value for a previously-obtained biological sample to generate a historical combined binding value.

The present disclosure also provides compositions comprising at least one first peanut peptide comprising the amino acid sequence WELQGDRRCQSQLER (SEQ ID NO:1) or an amino acid sequence comprising SEQ ID NO:1 but having one to four conservative amino acid substitutions therein, and at least one second peanut peptide comprising the amino acid sequence EYDEDEYEYDEEDRR (SEQ ID NO:2) or an amino acid sequence comprising SEQ ID NO:2 but having one to four conservative amino acid substitutions therein.

The present disclosure also provides kits for carrying out any of the methods described herein. In some embodiments, the kit comprises at least one first solid support coupled to at least one first peanut peptide comprising the amino acid sequence WELQGDRRCQSQLER (SEQ ID NO:1), or an amino acid sequence comprising SEQ ID NO:1 but having one to four conservative amino acid substitutions therein, and at least one second peanut peptide comprising the amino acid sequence EYDEDEYEYDEEDRR (SEQ ID NO:2) or an amino acid sequence comprising SEQ ID NO:2 but having one to four conservative amino acid substitutions therein. The kits also comprise at least one allergy associated immunoglobulin (AAI)-specific labeling reagent.

The kits described herein may also comprise additional components. In some embodiments, the kit further comprises instructions for use. In some embodiments, the kit further comprises at least one of at least one binding buffer, at least one wash buffer, at least one detection buffer, at least one non-allergic control sample, at least one negative buffer control sample, and at least one allergic positive control sample. In some embodiments, peptides containing non-reactive epitopes of peanut proteins can be used as negative controls.

The peanut peptides coupled to the solid support can be any of the peanut peptides described herein and can be coupled to the solid support by any of the means described herein. The solid supports can be any of the solid supports described herein. The at least one AAI-specific labeling reagents can be any of the at least one AAI-specific labeling reagent described herein. The at least one detectable label for any of the at least one AAI-specific labeling reagent can be any of the at least one detectable label described herein.

In some embodiments, the first amount of a peanut protein is about 444 mg of a peanut protein. In some embodiments, the second amount of a peanut protein is about 1444 mg of a peanut protein. In some embodiments, the first amount of a peanut protein is about 300 mg of a peanut protein. In some embodiments, the second amount of a peanut protein is about 1000 mg of a peanut protein.

In some embodiments, the at least one first biological sample comprises serum, plasma, saliva, or at least one buccal swab. In some embodiments, the at least one first biological sample comprises serum or plasma. In some embodiments, the at least one second biological sample comprises serum, plasma, saliva, or at least one buccal swab. In some embodiments, the at least one first biological sample comprises serum or plasma. In some embodiments, the at least one first biological sample and the at least one second biological sample are derived from the same biological sample.

In some embodiments, the at least one first peanut peptide is coupled to the at least one first solid support by at least one first linker-spacer. In some embodiments, the at least one second peanut peptide is coupled to the at least one second solid support by at least one second linker-spacer. In some embodiments, at least one of the at least one first linker-spacer and the at least one second linker-spacer comprises at least one linker comprising biotin, at least one thiol, hydrazine, and at least one amine. In some embodiments, at least one of the at least one second linker-spacer and the at least one second linker-spacer comprises at least one spacer comprising at least one polypeptide, at least one oligonucleotide, at least one alkyl group, or at least one polyethylene glycol (PEG) group. In some embodiments, the at least one spacer comprises at least one alkyl group or at least one PEG group. In some embodiments, the at least one alkyl group comprises at least one C₁-C₁₈alkyl group. In some embodiments, the at least one PEG group comprises at least one PEG group selected from PEG1 to PEG18 inclusive. In some embodiments, the at least one PEG group comprises PEG12. In some embodiments, at least one C-terminus of the at least one first peanut peptide is coupled to the at least one first solid support by the at least one first linker-spacer. In some embodiments, at least one C-terminus of the at least one second peanut peptide is coupled to the at least one solid support by the at least one second linker-spacer. In some embodiments, at least one N-terminus of the at least one first peanut peptide is coupled to the at least one first solid support by the at least one first linker-spacer. In some embodiments, at least one N-terminus of the at least one second peanut peptide is coupled to the at least one second solid support by the at least one second linker-spacer. In some embodiments, at least one C-terminus of the at least one peanut peptide is coupled to the at least one first solid support by at least one biotin-PEG12 linker-spacer.

In some embodiments, the at least one first solid support comprises at least one microsphere bead, at least one glass array, at least one silicone array, at least one membrane, or at least one microtiter plate. In some embodiments, the at least one second solid support comprises at least one microsphere bead, at least one glass array, at least one silicone array, at least one membrane, or at least one microtiter plate. In some embodiments, the at least one first microsphere bead and the at least one second microsphere bead are the same microsphere bead, the at least first one glass array and the at least second one glass array, the at least one first silicone array and the at least one second silicone array are the same silicone array, the at least one first membrane and the at least one second membrane are the same membrane, or the at least one first microtiter plate and the at least one second microtiter plate are the same microtiter plate. In some embodiments, the at least one first solid support is at least one first microsphere bead. In some embodiments, the at least one second solid support is at least one second microsphere bead. In some embodiments, the at least one first microsphere bead and the at least one second microsphere bead is the same microsphere bead. In some embodiments, the at least one first microsphere bead comprises at least one avidin-coupled microsphere bead. In some embodiments, the at least one second microsphere bead comprises at least one avidin-coupled microsphere bead.

In some embodiments, the at least one AAI-1 comprises at least one first IgM, at least one first IgA, and/or at least one first IgD. In some embodiments, the at least one AAI-2 comprises at least one second IgM, at least one second IgA, and/or at least one second IgD. In some embodiments, the at least one AAI-1 comprises at least one first IgG and/or at least one first IgE. In some embodiments, the at least one AAI-2 comprises at least one second IgG and/or at least one second IgE. In some embodiments, the at least one AAI-1 comprises at least one IgE. In some embodiments, the at least one AAI-2 comprises at least one IgE.

In some embodiments, the at least one AAI-1-specific labeling reagent comprises at least one first detectably labeled anti-human antibody. In some embodiments, the at least one AAI-2-specific labeling reagent comprises at least one second detectably labeled anti-human antibody. In some embodiments, the at least one first detectably labeled anti-human antibody comprises at least one first detectably labeled anti-human IgE antibody. In some embodiments, the at least one second detectably labeled anti-human antibody comprises at least one second detectably labeled anti-human IgE antibody. In some embodiments, the detectable label of the at least one first detectably labeled anti-human antibody comprises at least one first fluorophore. In some embodiments, the detectable label of the at least one second detectably labeled anti-human antibody comprises at least one second fluorophore. In some embodiments, the at least one first detectably labeled anti-human antibody and the at least one second detectably labeled anti-human antibody have the same chemical structure. In some embodiments, the first detectable label of the at least one first detectably labeled anti-human antibody comprises phycoerythrin (PE), at least one first cyanine dye, at least one first fluorescent dye, at least one first infrared dye, at least one first chromogenic dye, at least one first enzyme label, or at least one first radioactive label. In some embodiments, the second detectable label of the at least one second detectably labeled anti-human antibody comprises phycoerythrin (PE), at least one second cyanine dye, at least one second fluorescent dye, at least one second infrared dye, at least one second chromogenic dye, at least one second enzyme label, or at least one second radioactive label. In some embodiments, the first detectable label comprises PE. In some embodiments, the second detectable label comprises PE. In some embodiments, the at least one AAI-1-specific labeling reagent comprises at least one first PE-labeled anti-human IgE antibody. In some embodiments, the at least one AAI-1-specific labeling reagent comprises at least one second PE-labeled anti-human IgE antibody.

In some embodiments, the measuring of the binding of the AAI-1-specific labeling reagent to the at least one AAI-1-peptide-solid support complex is carried out by at least one first point of care device. In some embodiments, the measuring of the binding of the AAI-2-specific labeling reagent to the at least one AAI-2-peptide-solid support complex is carried out by at least one second point of care device. In some embodiments, the at least one first point of care device comprises at least one first multiplex peptide-bead flow cytometric analysis device or at least one first lateral flow assay device. In some embodiments, the at least one second point of care device comprises at least one second multiplex peptide-bead flow cytometric analysis device or at least one second lateral flow assay device. In some embodiments, the at least one first point of care device and at least one second point of care device are the same point of care device.

In some embodiments, the at least one AAI-1-specific labeling reagent comprises at least one first fluorophore; the at least one AAI-2-specific labeling reagent comprises at least one second fluorophore; the measuring the binding of the at least one AAI-1-specific labeling reagent to the at least one AAI-1-peptide-solid support complex comprises measuring a first median fluorescent intensity (MFI-1₁), a second median fluorescent intensity (MFI-1₂), and a third median fluorescent intensity (MFI-1₃) of the at least one first fluorophore; the measuring the binding of the at least one AAI-2-specific labeling reagent to the at least one AAI-2-peptide-solid support complex comprises measuring a first median fluorescent intensity (MFI-2₁), a second median fluorescent intensity (MFI-2₂), and a third median fluorescent intensity (MFI-2₃), of the at least one second fluorophore, generating of the combined binding value comprises: determining a transformed MFI-1 (tMFI-1), where: tMFI-1=log₂(median(MFI-1₁, MFI-1₂, MFI-1₃)+1);

$$tMFI - 1 = \{ \begin{matrix}tMFI - 1 , & if tMFI - 1 \geq LOD - 1 \\ 0 , & if tMFI - 1 < LOD - 1\end{matrix} ,$$

and where LOD-1 signifies a first limit of detection of about 2.4; determining a transformed MFI-2 (tMFI-2) where: tMFI-2=log₂(median(MFI-2₁, MFI-2₂, MFI-2₃)+1);

$$tMFI - 1 = \{ \begin{matrix}tMFI - 2 , & if tMFI - 2 \geq LOD - 2 \\ 0 , & if tMFI - 2 < LOD - 2\end{matrix} ,$$

and where LOD-2 signifies a first limit of detection of about 2.4; determining a calibrated MFI-1 (calMFI-1) from the tMFI-1, where:

$$calMFI - 1 \{ \begin{matrix}first correction factor * tMFI - 1 , \\ if tMFI - 1 > 0 , \\ 0 , if tMFI - 1 \leq 0\end{matrix}$$

and where the first correction factor is as described herein; determining a calibrated MFI-2 (calMFI-2) from the tMFI-2, where:

$$calMFI - 2 \{ \begin{matrix}second correction factor * tMFI - 2 , \\ if tMFI - 2 > 0 , \\ 0 , if tMFI - 2 \leq 0 ; and\end{matrix}$$

determining a predicted score, where: Predicted score=6.82603−(0.229755*calMFI-1)−(0.134746*calMFI-2); wherein the predicted score relates to the cumulative reactive dose.

In some embodiments, the at least one AAI-1-specific labeling reagent comprises at least one first fluorophore; the at least one AAI-2-specific labeling reagent comprises at least one second fluorophore; the measuring the binding of the at least one AAI-1-specific labeling reagent to the at least one AAI-1-peptide-solid support complex comprises measuring a first net median fluorescent intensity (Net MFI-1₁), a second net median fluorescent intensity (Net MFI-1₂), and a third net median fluorescent intensity (MFI-1₃) of the at least one first fluorophore; the measuring the binding of the at least one AAI-2-specific labeling reagent to the at least one AAI-2-peptide-solid support complex comprises measuring a first net median fluorescent intensity (Net MFI-2₁), a second net median fluorescent intensity (MFI-2₂), and a third net median fluorescent intensity (Net MFI-2₃), of the at least one second fluorophore, generating a Positive Control NetMFI-1, where: Positive Control Net MFI-1=log₂(Median(P-1ᵣ₁, P-1ᵣ₂, P-1ᵣ₃)+1) and where P-1ᵣ₁ is the numerical value of Net MFI-1₁, P-1ᵣ₂ is the numerical value of Net MFI-1₂, and P-1ᵣ₂ is the numerical value of Net MFI-1ₛ; generating a Positive Control NetMFI-2, where: Positive Control Net MFI-2=log 2(Median(P-2ᵣ₁, P-2ᵣ₂, P-2ᵣ₃)+1); and where P-2ᵣ₁ is the numerical value of Net MFI-2₁, P-2ᵣ₂ is the numerical value of Net MFI-2₂, and P-2ᵣ₂ is the numerical value of Net MFI-1ₛ; generating a MedianNetMFI-1, where:

$$MedianNetMFI - 1 \{ \begin{matrix}MedianNetMFI - 1 , & if MedianNetMFI - 1 \\ 0 , & if MedianNetMFI - 1\end{matrix} ;$$

generating a MedianNetMFI-2, where:

$$MedianNetMFI - 2 \{ \begin{matrix}MedianNetMFI - 2 , & if MedianNetMFI - 2 \\ 0 , & if MedianNetMFI - 2\end{matrix} ;$$

generating a NetMFI-1, where NetMFI-1=log₂(MedianNetMFI-1)+1); generating a NetMFI-2, where NetMFI-2=log₂(MedianNetMFI-2)+1); generating a Correction Factor-1, where the Correction Factor-1 is the quotient of 6.6865 and the Positive Control NetMFI-1; generating a Correction Factor-2, where the Correction Factor-2 is the quotient of 7.6439 and the Positive Control NetMFI-2; generating a Calibrated NetMFI-1, where:

$$\begin{matrix}Calibrated \\ NetMFI - 1\end{matrix} \{ \begin{matrix}Correction Factor - 1 * NetMFI - 1 , & if NetMFI - 1 \\ 0 , & if MedianNetMFI - 1\end{matrix} ;$$

generating a Calibrated NetMFI-2, where:

$$\begin{matrix}Calibrated \\ NetMFI - 2\end{matrix} \{ \begin{matrix}Correction Factor - 2 * NetMFI - 2 , & if NetMFI - 2 \\ 0 , & if MedianNetMFI - 2\end{matrix} ;$$

generating a Calibrated NetMFI-1, where:

$$\begin{matrix}Calibrated \\ NetMFI - 1\end{matrix} \{ \begin{matrix}Correction Factor - 1 * NetMFI - 1 , \\ if NetMFI - 1 \geq LOD - 1 \\ 0 , if MedianNetMFI - 1 < LOD - 1\end{matrix} ;$$

generating a Calibrated NetMFI-2, where:

$$\begin{matrix}Calibrated \\ NetMFI - 2\end{matrix} \{ \begin{matrix}Correction Factor - 2 * NetMFI - 2 \\ if NetMFI - 2 \geq LOD - 2 \\ 0 , if MedianNetMFI - 2 < LOD - 2\end{matrix} ,$$

and where LOD-1 and LOD-2 both equal log₂(2.4+1); and generating a Prediction Score, where:

$$\begin{matrix}Prediction \\ Score\end{matrix} \{ \begin{matrix}4.8542 + 0.0446 * Calibrated NetMFI - 1 - 0.1148 \\ * Calibrated NetMFI - 2\end{matrix} ;$$

wherein the Prediction Score relates to the CRD.

In some embodiments, at least one of the first median fluorescent intensity (MFI-1₁), the second median fluorescent intensity (MFI-1₂), and the third median fluorescent intensity (MFI-1₃) is background subtracted. In some embodiments, at least one of the first net median fluorescent intensity (Net MFI-1₁), the second net median fluorescent intensity (Net MFI-1₂), and the third median fluorescent intensity (Net MFI-1₃) is background subtracted.

In some embodiments, the at least one first peanut peptide is administered before the at least one second peanut peptide. In some embodiments, the at least one first peanut peptide is administered after the at least one second peanut peptide. In some embodiments, the at least one first peanut peptide is administered at the same time as the at least one second peanut peptide.

In some embodiments, the at least one first peanut peptide and the at least one second peptide are disposed in a pharmaceutical composition.

In some embodiments, the composition comprises at least one first peanut peptide comprising the amino acid sequence WELQGDRRCQSQLER (SEQ ID NO:1) or an amino acid sequence comprising SEQ ID NO:1 but having one to four conservative amino acid substitutions therein and at least one second peanut peptide comprising the amino acid sequence EYDEDEYEYDEEDRR (SEQ ID NO:2) or an amino acid sequence comprising SEQ ID NO:2 but having one to four conservative amino acid substitutions therein. In some embodiments, the at least one first peanut peptide is coupled to at least one first solid support. In some embodiments, the at least one second peanut peptide is coupled to at least one second solid support. In some embodiments, the at least one first peanut peptide is coupled to the at least one first solid support by at least one first linker-spacer. In some embodiments, the at least one second peanut peptide is coupled to the at least one second solid support by at least one second linker-spacer. In some embodiments, the at least one first linker-spacer comprises at least one linker comprises at least one first biotin, at least one first thiol, at least one first hydrazine, and at least one first amine. In some embodiments, the at least one second linker-spacer comprises at least one second linker comprises biotin, at least one second thiol, at least one second hydrazine, and at least one second amine. In some embodiments, the at least one first linker-spacer comprises at least one first spacer comprising at least one first polypeptide, at least one first oligonucleotide, at least one first alkyl group, or at least one first polyethylene glycol (PEG) group. In some embodiments, the at least one second linker-spacer comprises at least one second spacer comprising at least one second polypeptide, at least one second oligonucleotide, at least one second alkyl group, or at least one second polyethylene glycol (PEG) group. In some embodiments, the at least one first spacer comprises at least one first alkyl group or at least one first PEG group. In some embodiments, the at least one second spacer comprises at least one second alkyl group or at least one second PEG group. In some embodiments, the at least one first alkyl group comprises at least one first C₁-C₁₈alkyl group. In some embodiments, the at least one second alkyl group comprises at least one second C₁-C₁₈alkyl group. In some embodiments, the at least one first PEG group comprises at least one first PEG1 to at least one first PEG18. In some embodiments, the at least one second PEG group comprises at least one second PEG1 to at least one second PEG18. In some embodiments, the at least one first PEG group comprises at least one first PEG12. In some embodiments, the at least one second PEG group comprises at least one second PEG12. In some embodiments, at least one first C-terminus of the at least one first peanut peptide is coupled to the at least one first solid support by the at least one first linker-spacer. In some embodiments, at least one second C-terminus of the at least one second peanut peptide is coupled to the at least one second solid support by the at least one second linker-spacer. In some embodiments, at least one first N-terminus of the at least one first peanut peptide is coupled to the at least one first solid support by the at least one first linker-spacer. In some embodiments, at least one second N-terminus of the at least one second peanut peptide is coupled to the at least one second solid support by the at least one second linker-spacer. In some embodiments, at least one first C-terminus of the at least one first peanut peptide is coupled to the at least one first solid support by at least one first biotin-PEG12 linker-spacer. In some embodiments, at least one second C-terminus of the at least one second peanut peptide is coupled to the at least one second solid support by at least one second biotin-PEG12 linker-spacer.

In some embodiments of the compositions, the at least one first solid support comprises at least one first microsphere bead, at least one first glass array, at least one first silicone array, at least one first membrane, or at least one first microtiter plate. In some embodiments, the at least one second solid support comprises at least one second microsphere bead, at least one second glass array, at least one second silicone array, at least one second membrane, or at least one second microtiter plate. In some embodiments, the at least one first solid support comprises at least one first microsphere bead. In some embodiments, the at least one second solid support comprises at least one second microsphere bead. In some embodiments, the at least one first microsphere bead comprises at least one first avidin-coupled microsphere bead. In some embodiments, the at least one second microsphere bead comprises at least one second avidin-coupled microsphere bead.

In some embodiments of the kits, at least one first solid support coupled to at least one first peanut peptide comprising the amino acid sequence WELQGDRRCQSQLER (SEQ ID NO:1) or an amino acid sequence comprising SEQ ID NO:1 but having one to four conservative amino acid substitutions therein; at least one second solid support coupled to at least one second peanut peptide comprising the amino acid sequence EYDEDEYEYDEEDRR (SEQ ID NO:2) or an amino acid sequence comprising SEQ ID NO:2 but having one to four conservative amino acid substitutions therein; and at least one allergy associated immunoglobulin (AAI)-specific labeling reagent. In some embodiments, the kit further comprising instructions for use. In some embodiments, the kit further comprises at least one of at least one binding buffer, at least one wash buffer, at least one detection buffer, at least one non-allergic control sample, at least one negative buffer control sample, and at least one allergic positive control sample. In some embodiments, the at least one first peanut peptide is coupled to the at least one first solid support by at least one linker-spacer. In some embodiments, at least one second peanut peptide is coupled to the at least one second solid support by at least one second linker-spacer. In some embodiments, at least one of the at least one first linker-spacer and the at least one second linker-spacer comprises at least one linker comprising at least one of at least one biotin, at least one thiol, at least one hydrazine, and at least one amine. In some embodiments, at least one of the at least one first linker-spacer and the at least one second linker-spacer comprises at least one spacer comprising at least one polypeptide, at least one oligonucleotide, at least one alkyl group, or at least one polyethylene glycol (PEG) group. In some embodiments, the at least one spacer comprises at least one alkyl group or at least one PEG group. In some embodiments, the at least one alkyl group comprises at least one C₁-C₁₈alkyl group. In some embodiments, the at least one PEG group comprises at least one PEG1 to at least one PEG18. In some embodiments, the at least one PEG group comprises at least one PFG12. In some embodiments, the C-terminus of at least one of the at least one first peanut peptide and the at least one second peanut peptide is coupled to at least one of the at least one first solid support and the at least one second solid support by the at least one linker-spacer. In some embodiments, the N-terminus of at least one of the at least one first peanut peptide and the at least one second peanut peptide is coupled to at least one of the at least one first solid support and the at least one second solid support by the at least one linker-spacer. In some embodiments, the C-terminus of at least one of the at least one first peanut peptide and the at least one second peanut peptide is coupled to at least one of the at least one first solid support and at least one second solid support by at least one biotin-PEG12 linker-spacer. In some embodiments, at least one of the at least one first solid support and the at least one second solid support comprises at least one microsphere bead, at least one glass array, at least one silicone array, at least one membrane, or at least one microtiter plate. In some embodiments, at least one of the at least one first solid support and the at least one second solid support comprises at least one microsphere bead. In some embodiments, the at least one microsphere bead comprises at least one avidin-coupled microsphere bead. In some embodiments, the at least one AAI-specific labeling reagent comprises at least one detectably labeled anti-human antibody. In some embodiments, the at least one detectably labeled anti-human antibody comprises at least one detectably labeled anti-human IgE antibody. In some embodiments, the at least one AAI-specific labeling reagent comprises at least one detectable label and the at least one detectable label comprises phycoerythrin (PE), at least one cyanine dye, at least one fluorescent dye, at least one infrared dye, at least one chromogenic dye, at least one enzyme label, and at least one radioactive label. In some embodiments, the at least one detectable label comprises PE. In some embodiments, the at least one AAI-specific labeling reagent comprises at least one PE-labeled anti-human IgE antibody. In some embodiments, the kit further comprises at least one reporter moiety capable of binding specifically to the at least one AAI-specific labeling reagent. In some embodiments, the at least one first solid support and the at least one second solid support are the same solid support.

In order that the subject matter disclosed herein may be more efficiently understood, examples are provided below. It should be understood that these examples are for illustrative purposes only and are not to be construed as limiting the claimed subject matter in any manner.

### EXAMPLES

### Example 1: Cumulative Reactive Dose for Peanut Allergic Children Using Epitope Profiling

### Methods:

During the discovery phase, patients' samples were obtained from baseline/enrollment DBPCFC of the clinical trials titled Immune and Clinical Implications of Threshold-based Phenotypes of Peanut Allergy (CAFETERIA) (ClinicalTrials.gov Identifier NCT03907397), Peanut Epicutaneous Phase II Immunotherapy Clinical Trial (CoFAR6) (ClinicalTrials.gov Identifier No. NCT01904604; Jones et al., J. Allergy Clin. Immunol., 2017, 139, 1242-1252), and Efficacy and Safety of Viaskin Peanut in Children With Immunoglobulin E (IgE)—Mediated Peanut Allergy (PEPITES) (ClinicalTrials.gov Identifier NCT02636699; Fleischer et al., J. Amer. Med. Assoc., 2019, 321, 946-955). Together these samples comprise the development cohort.

Epitope-specific IgE quantification: samples from the CAFETERIA, CoFAR6, and PEPITES trials were randomized across 96-well plates using PlateDesigner (Suprun et al., Bioinformatics, 2019, 35, 1605-1607). A bead-based epitope assay (BBEA) was carried out as described previously (see, Suirez-Farinas, Mech. Allergy/Immunol., 2021, 148, 835-842). IgE antibodies to 64 sequential epitopes from three peanut proteins (Ara h 1 (n=34), Ara h 2 (n=16), and Ara h 3 (n=14)) were quantified. The amino acid sequences of the three peanut proteins are published elsewhere (see, Suirez-Farinas, Mech. Allergy/Immunol., 2021, 148, 835-842 and Suprun et al., J. Allergy Clin. Immunol., 2020, 146, 1080-1088). In brief, 15-mer biotinylated peptides were coupled to LUMAVIDIN® microspheres (Luminex Corporation) and this master mix was added to 96-well filter plates. Every plate also included three peptide-only wells for background quantification (NEG) and three wells with a positive control sample (POS) for the downstream calibration across all plates. After three washes, 100 μL/well of 1:10 diluted plasma or serum samples were added in triplicate and incubated with the peptides for 2 hours. After two additional washes, samples were incubated with 50 μL/well of mouse anti-human phycoerythrin (PE) conjugated IgE (Thermo-Pierce Antibodies, Clone BE5, 1:50 dilution) for 30 minutes. Plates were read on a Luminex-200 instrument (Luminex Corporation) and IgE for each sample and epitope quantified as a Median Fluorescence Intensity (MFI).

For each sample, the median MFI was transformed for each sample (s) and epitope-specific IgE (e) as:

$${tMFI}_{s , e} = {log}_{2} ( median ( {MFI}_{{replicate}_{{1}_{s , e}}} , {MFI}_{{replicate}_{{2}_{s , e}}} , {MFI}_{{replicate}_{{3}_{s , e}}} ) + 1 ) ;$$

$${tMFI}_{s , e} = \{ \begin{matrix}{tMFI}_{s , e} , & if {tMFI}_{s , e} \geq LOD \\ 0 , & if {tMFI}_{s , e} < LOD\end{matrix} ,$$

where LOD is a limit of detection of 2.4 (determined from the NEG wells) (Suarez-Farinas et al., Allergy, 2021, 76, 3789-3797). The LOD value was the same for all 64 epitopes. Next, and to ensure that the median MFI values were comparable across plates, an epitope-specific “correction factor” was determined using the POS sample on each plate. The correction factors were determined by taking the median value of all positive control (calibration samples) used in the study. There is a ‘Universal Calibration’ value for each analyte. It is based on the median of the calibrator control value across the sample plates used in the discovery process. It is the same method that was used in P.DIA.1. Alternately, all the median log adjusted netMFI values from the new Calibrator batch were collected. The calibrated MFI was computed as:

$${calMFI}_{s , e} = \{ \begin{matrix}correction {factor}_{e} * {tMFI}_{s , e} , & if {tMFI}_{s , e} > 0 , \\ 0 , & if {tMFI}_{s , e} \leq 0\end{matrix} .$$

Determination and evaluation of the prediction rule: For each pair of sequential epitope-specific IgEs (ses-IgE) (2016 combinations from 64 epitopes chosen in pairs with no repeats), a linear regression was fitted to predict the natural logarithm of the CRD of the Discovery cohort.

### Calcuation

$${C}_{k} ( n ) = ( \begin{matrix}n \\ k\end{matrix} ) = \frac{n !}{k ! ( n - k ) !}$$

$$n = 64$$

$$k = 2$$

$${C}_{2} ( 64 ) = ( \begin{matrix}64 \\ 2\end{matrix} ) = \frac{64 !}{2 ! ( 64 - 2 ) !} = \frac{64 \cdot 63}{2 \cdot 1} = 2016$$

$$Number of combinations : 2016$$

Pearson (r) and Spearman (rho) correlations were used to measure the linear correlation between the score predicted by each ses-IgE pair and the actual CRD levels of the patients. After the best model was identified, which included IgE to Ara h 2_008 and Ara h 3_100 epitopes, the best model was documented and locked before evaluation of that model's performance on the Validation samples. The locked down model was selected by picking the pair of epitopes whose model had the highest coefficient of determination R² in the discovery dataset.

Using the Validation samples, the algorithm was applied to predict CRDs. Using the predicted CRD, patients were split into 3 groups: “≤444”, “444−≤1444”, and “≥1444” mg of peanut protein. Within each group, the proportion of subjects that reacted at 4, 14, 44, 144, 444, and 1444 mg was calculated.

Mapping of epitopes to crystal structures: Amino acid sequences of Aa h 2_008 and Ara h 3_100 epitopes are mapped to the crystal structures of Ara h 2 (30B4) and Ara h 3 (3C₃V) proteins using the PYMOL® software.

### Results

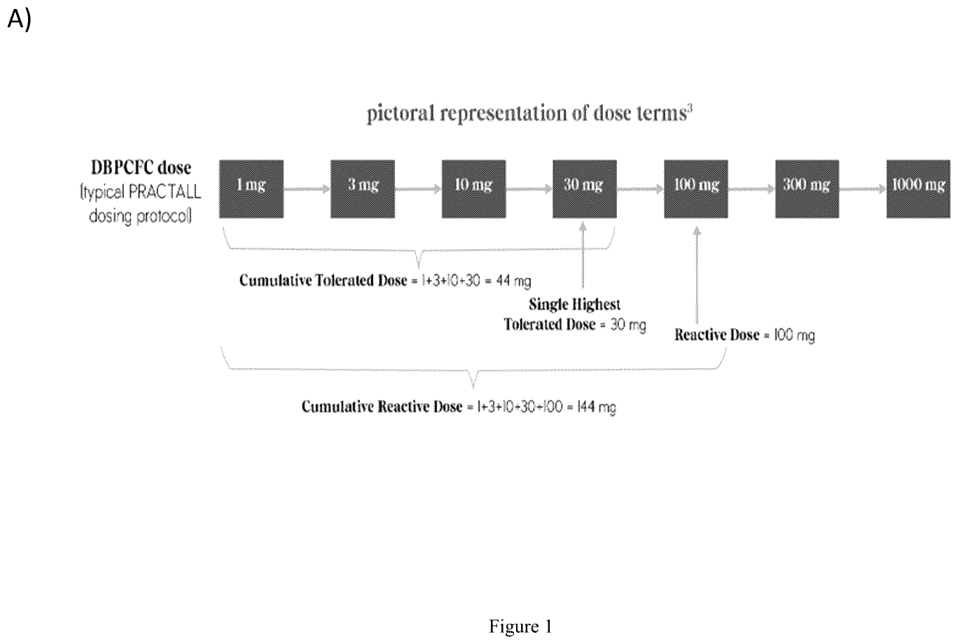

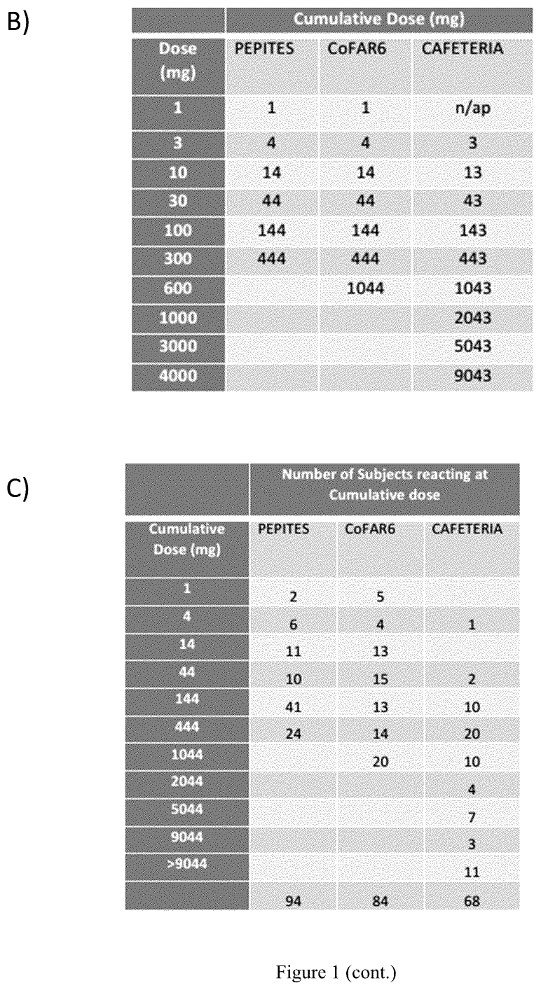

Study design and cohorts: A schematic of a DBPCFC dosing protocol and the specific protocols that were used in the cohorts are depicted in FIG. 1. Two hundred and forty-six subjects with available DBPCFC data from three independent cohorts: CoFAR6 (n=84), CAFETERIA (n=68), and PEPITES (n=94). The participants' ages ranged across cohorts from 4 to 25 years of age. The DBPCFC protocols varied by study but followed PRACTALL guidelines for incremental dose increase (see, Sampson).

In adherence with the guidelines by the National Academy of Medicine, the CRD algorithm was validated on samples not used in the discovery work. The Validation cohort included 152 subjects from the CoFAR6 and CAFETERIA trials. Additionally, all the subjects from the PEPITES trial were used for a final testing of the algorithm.

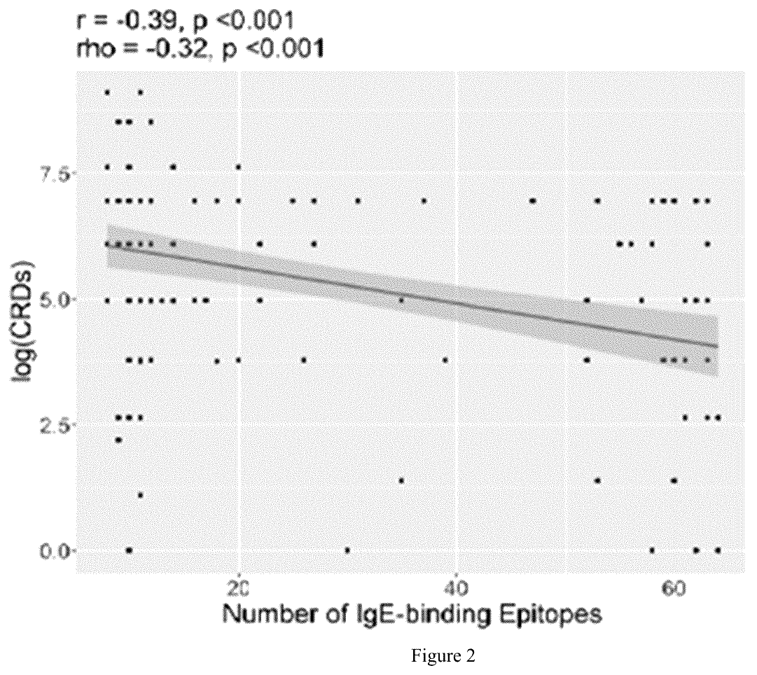

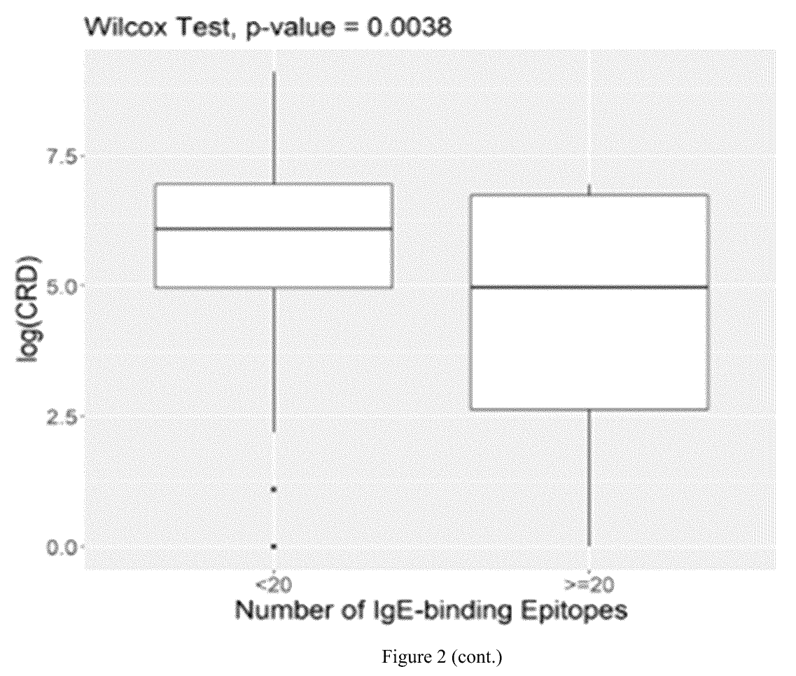

Ses-IgEs are associated with CRDs: Patients reacting at lower CRDs were generally observed to have a greater number of epitopes recognized by IgE antibodies (see, FIG. 2). The number of epitopes, with a calibrated MFI value greater than 2.5, increase as a subjects tolerance to peanut protein decreases as reflected by lower CRD values. When subjects are segregated by the number of IgE-binding epitopes, into less than or more than 20, the CRD values can be seen as statistically significantly distinct populations (Wilcox Test p-value of 0.0038. In this regard, several studies have showed that IgE diversity (“epitope spreading,” i.e., recognition of a greater number of epitopes) is associated with negative outcomes such as more severe allergic reactions or a persistent disease phenotype. A moderate negative correlation between the number of IgE epitopes and CRD was observed (rho=−0.32, p<0.001). This suggests that a higher IgE diversity is associated with a lower amount of peanut antigens a patient can consume.

Individual ses-IgE antibodies were strongly correlated among themselves (median rho=0.85 (0.76, 0.87)), as we have observed in several other studies. Of all ses-IgEs, 61 (95%) were negatively correlated with CRDs (see, FIG. 2, Panel C). However, those associations were variable, ranging from rho=−0.14 to −0.55, suggesting that IgE-binding epitopes have varying impact on the amount of peanut a patient can consume. These unexpected findings suggested that a combination of at least two such epitopes can be a stronger predictor of CRD.

An epitope-based algorithm predicts CRD: A pair of ses-IgEs that together provided the best prediction of CRD were identified, namely ara h 2_008 and ara h 3_100. A prediction rule for each sample (s) using only the Discovery cohort was devised:

$$Predicted {Score}_{( s )} = 6.82603 - ( 0.229755 * {calMFI}_{Ara h 2 \_ 008 ( s )} ) - ( 0.134746 * {calMFI}_{Ara h 3 \_ 100 ( s )} )$$

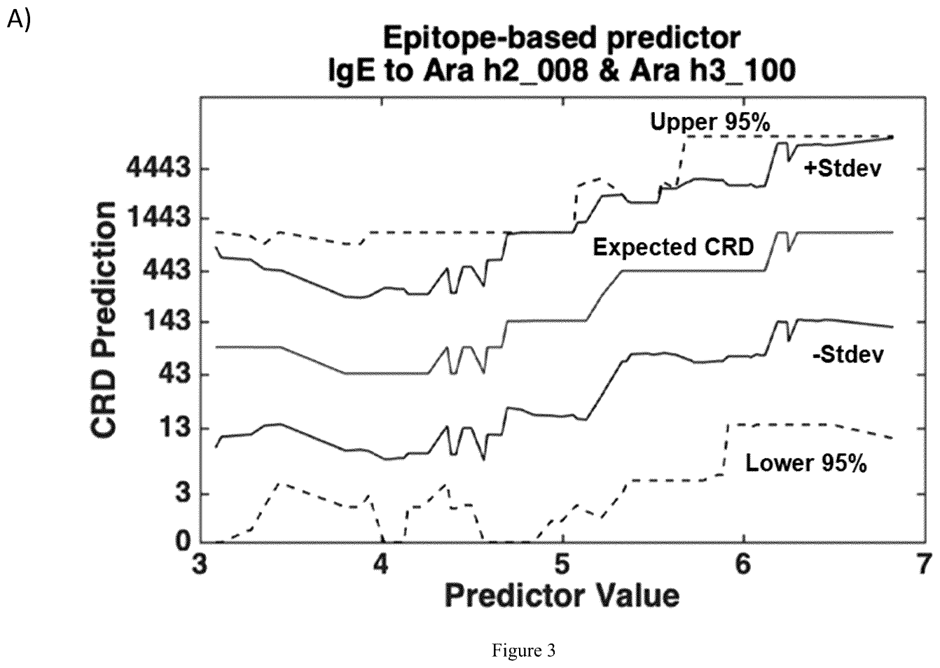

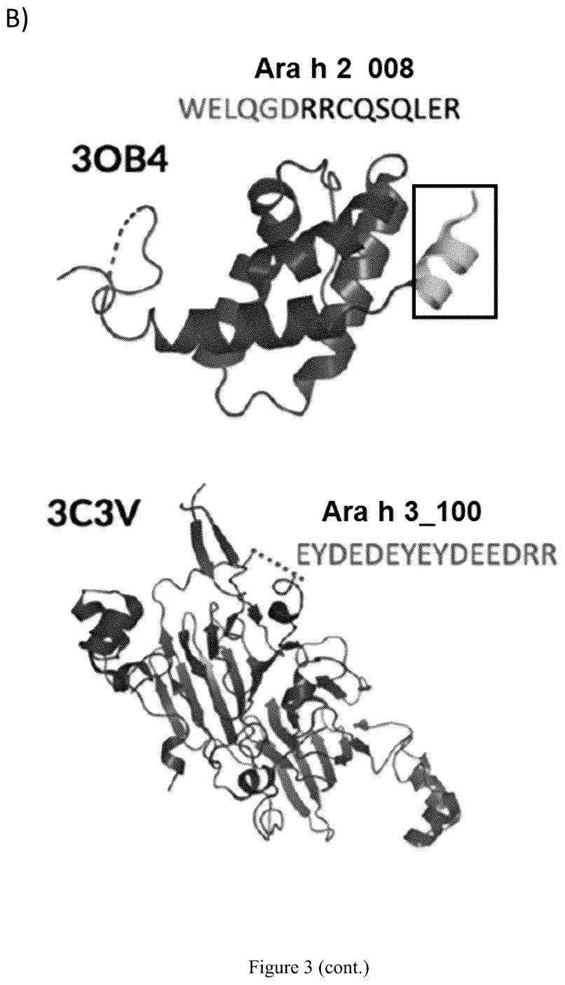

For the IgE-binding epitopes ara h 2_008 and ara h 3_100, the correlation of the predicted score with CRD was rho=0.48 (p≤0.001) in the Validation cohort. Importantly, the predicted score increased incrementally with the increase in CRD (see, FIG. 3, Panel A).

This algorithm was then documented, locked, and validated on 201 subjects the data for whom had never been used in any aspect of the discovery process. As expected, the performance of the algorithm was lower with a Spearman correlation of 0.51 (p≤0.001).

The two IgE-binding epitopes that were included in the algorithm were from the Ara h 2 and Ara h 3 proteins, namely Ara h 2_008 and Ara h 3_100. FIG. 3, Panel B shows amino acid sequences and positions on conformational protein structures for both epitopes. Interestingly, both epitopes do not have a complete structure resolved, with 9/15 amino acids available for Ara h 2 008, and none for Ara h 3_100.

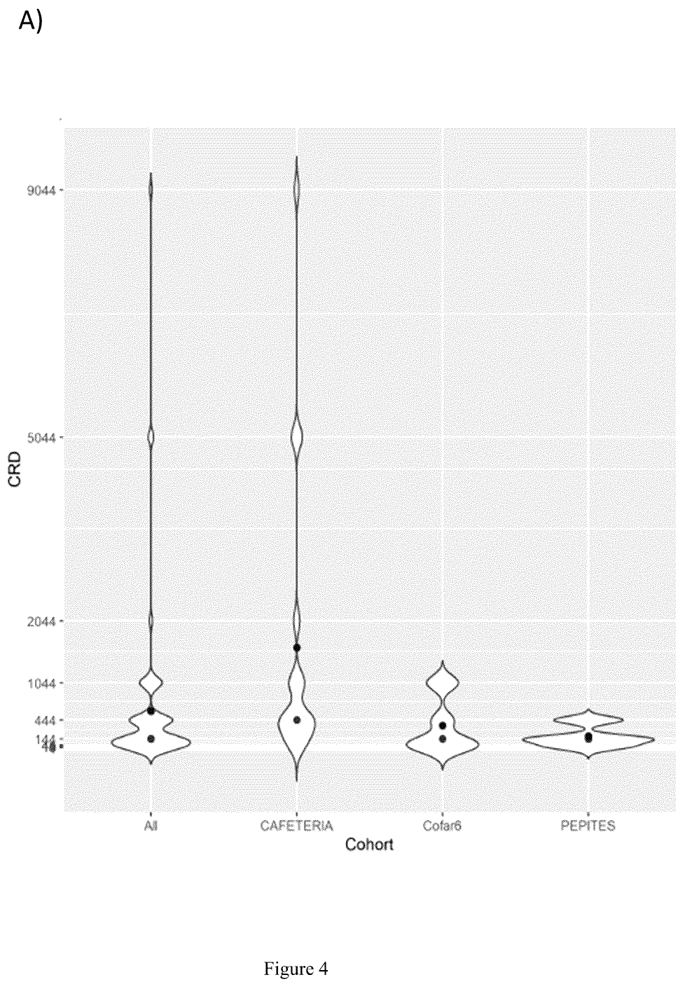

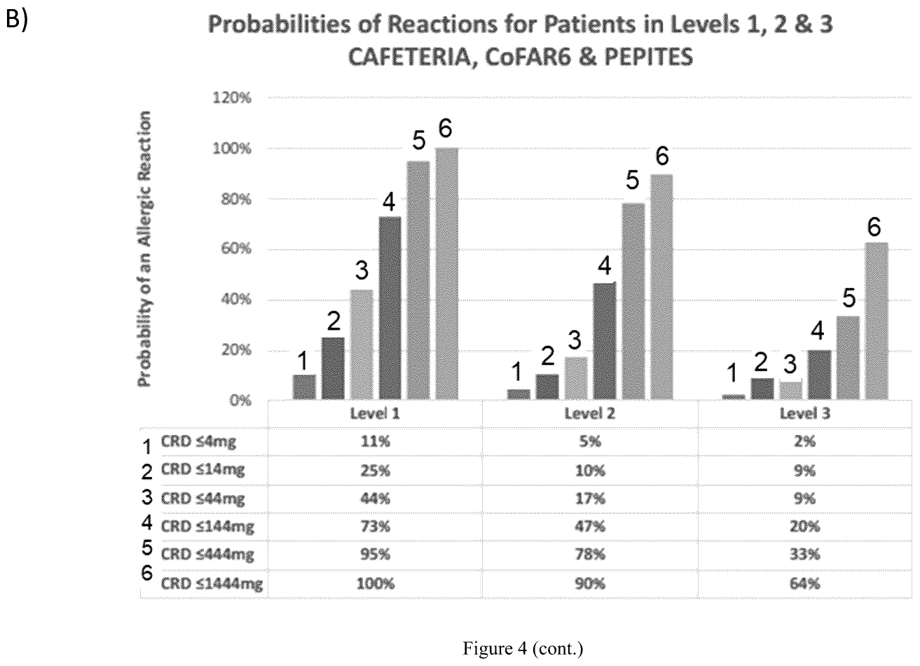

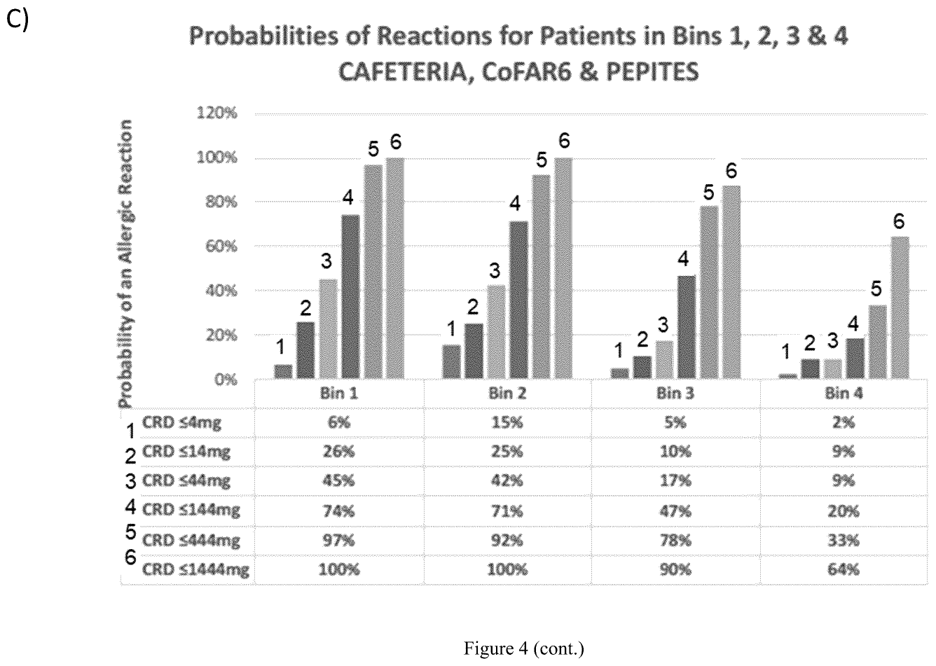

The ses-IgE-based algorithm predicts CRD: Since CRDs are not truly continuous, CRDs generally increase 3-fold at each of the escalation doses, and the sample size at each dose tends to be small (FIG. 4, Panel A), a large number of subjects would normally need to undergo DBPCFCs to devise a predictive rule with a high correlation with CRDs. To address this issue and to allow flexibility for the predictive algorithm as more data become available, subjects were separated into four bins; Bin 1 (≤100 mg), Bin 2 (100 mg-≤300 mg), Bin3 (300 mg-≤1000 mg) and Bin 4 (>1000 mg). Based on analysis of sample numbers and reactivity profiles, Bins were translated into 3 levels of sensitivity/reactivity: Level 1 (high level of sensitivity/reactivity) combines Bins 1 and 2, Level 2 (moderate sensitivity/reactivity) aligns with Bin 3 and Level 3 (low sensitivity/reactivity) aligns with Bin 4. The three CRD groups or “levels,” namely “≤444” (n=73; “Level 1), “444−≤1444” (n=77; “Level 2”), and “≥1444” (n=51; “Level 3”) mg of peanut protein, using the ses-IgE-based predictor's CRD assignment output.

For each group, the proportion of subjects that would react at each discrete CRD was calculated (see, FIG. 4, Panel B). On average, subjects in the least sensitive group Level 3) were on average 3.4 times more likely to tolerate a specific dose, compared to the most sensitive group (Level 1):

TABLE 1

| | Level 1/Level 3 |
|---|---|
| | (Probability of |
| | Allergic Reaction) |
| 4 mg | 4.7 |
| 14 mg | 2.9 |
| 44 mg | 4.9 |
| 144 mg | 3.6 |
| 444 mg | 2.8 |
| 1444 mg | 1.6 |
| Average | 3.4 |

For example, the predicted probabilities of reacting to cumulative doses of 4, 44, 444 and 1444 mg in the most sensitive group (Level 1) were 11%, 44%, 95%, and 100% compared to 2%, 9%, 33%, and 64% in the least sensitive group (Level 3), respectively. In light of the present disclosure but not before, the CRD can be converted and interpreted as other types of DBPCFC outcomes. For example, instead of presenting probabilities of reactions at cumulative doses of 4, 44, 444 and 1444 mg, the algorithm's output can offer a probability of successfully consuming a Single Highest Tolerated Dose (SHTD) of 1, 10, 100, and 300 mg or of successfully tolerating a cumulative dose of 1, 14, 144 and 444 mg:

TABLE 2

| | | Cumulative | |
|---|---|---|---|
| | Dose (mg) | Dose (mg) | |
| | 1 | 1 | |
| | 3 | 4 | |
| | 10 | 14 | |
| 10 mg - SHTD −> | 30 | 44 | <− 14 mg - CTD |
| 30 mg Reactive | STOP (patient | | <− 44 mg - CRD |
| Dose −> | reacted) | | |

In view of the disclosed algorithm, the blood work of a patient seeking an allergy evaluation should include measurements of IgE levels with respect to two epitopes (Ara h 2_008 and Ara h 3_100). After the ses-IgE is quantified, the predictive algorithm can assign a patient to one of the three groups (Levels 1, Level 2, or Level 3) and a clinician will be provided a patient-specific probability of having a reaction at each cumulative dose or with a probability of tolerating each cumulative dose. When patients were divided into 4 CRD bins by splitting the “≤444” into “1−≤144” and “144−≤444” mg, this further separation did not offer any improvement of the results (see, FIG. 4, Panel C).

TABLE 3

| | Bin 1/Bin 4 |
|---|---|
| | (Probability of |
| | Allergic Reaction) |
| 4 mg | 2.9 |
| 14 mg | 2.9 |
| 44 mg | 5.1 |
| 144 mg | 3.7 |
| 444 mg | 2.9 |
| 1444 mg | 1.6 |
| Average | 3.18 |

TABLE 4

| | # Patients in |
|---|---|
| Assigned Level | Assigned Bin |
| Bin 1 | 62 |
| Bin 2 | 52 |
| Bin 3 | 87 |
| Bin 4 | 45 |

### Example 2: P.RT. Reactivity Tests

Introduction: The P.RT. Reactivity test (“Test”) is a BBEA developed to utilize blood epitope levels to evaluate the amount of peanut protein a peanut-allergic subject can tolerate. The epitopes employed by the algorithm to generate test results are ara h 2_008 and ara h 3_100. The BBEA was conducted as disclosed in Example 1.

The Test generates a predictive value that correlates with CRD identified through an OFC. The predictive value was used to assign each subject to one of four CRD “bins.” The CRD bins, and boundaries associated therewith, were determined using sample data from clinical trials following OFCs (see, Sampson).

The assigned CRD bin indicates a subject's level of reactivity to peanut proteins. A subject assigned to Bin 1 or 2 is considered to have “Level 1” reactivity, namely a high level of reactivity to peanut protein (low tolerance). The mapping of assigned bins to reactivity levels can be seen in Table 5:

TABLE 5

| Bin | CRD Range (mg) | | Reactivity Level |
|---|---|---|---|
| Bin 1 | <100 | mg | Level 1 (high) |
| Bin 2 | 100 to <300 | mg | |
| Bin 3 | 300 to <1000 | mg | Level 2 (moderate) |
| Bin 4 | ≥1000 | mg | Level 3 (low) |

Table 5 represents the binning of CRD values and the associated subjects' level of reactivity to peanut protein.

The performance of the Test was evaluated on 246 validation samples from peanut-allergic subjects, the results of which are described in the following sections.

Samples: Two hundred and forty-six samples from three study cohorts of peanut-allergic subjects were used to evaluate the performance of the Test: CAFETERIA (68 plasma samples), CoFAR6 (84 plasma samples), and PEPITES (94 serum samples). Each subject had a known CRD value derived through an OFC. The doses and cumulative doses generated by the OFC protocols for each study can be seen in Table 6:

TABLE 6

| | |
|---|---|
| Dose | Cumulative Dose (mg) |

| Bin | (mg) | CAFETERIA | CoFAR6 | PEPITES |
|---|---|---|---|---|
| 1 | 1 | | 1 | 1 |
| | 3 | 3 | 4 | 4 |
| | 10 | 13 | 14 | 14 |
| | 30 | 43 | 44 | 44 |
| 2 | 100 | 143 | 144 | 144 |
| 3 | 300 | 443 | 444 | 444 |
| | 600 | 1043 | 1044 | |
| 4 | 1000 | 2043 | | |
| | 3000 | 5043 | | |
| | 4000 | 9043 | | |

Table 6 represents the dosing protocols used in the CAFETERIA, CoFAR6, and PEPITES trials from which the validation samples were obtained and the cumulative doses to which such validation samples correspond. The first dose used in the CoFAR6 and PEPITES trials was 1 mg while that in the CAFETERIA trial was 3 mg. As such, the cumulative doses in the samples of the CAFETERIA trials are all 1 mg less than the equivalent doses in the CoFAR6 and PEPITES trials.

The 246 validation subjects were segregated into one of four bins based on their OFC-derived CRDs. The performance of the Test was then evaluated in terms of how accurately the Test predicted the bin.

Table 7 shows the number of subjects from each of the three cohorts that fall into Bins 1 to 4. In this regard, the CAFETERIA and CoFAR6 trials included a 600 mg single dose in their OFCs that is not part of the PRACTALL progression. The subjects for whom the 600 mg single dose was a reactive dose therefore have non-PRACTALL CRDs of 1043/1044. These non-PRACTALL CRDs aligned most closely to the values in bin 3 and were therefore included in that bin.

TABLE 7
CRD-

| based | | CRD Range | | CAFE- | | |
|---|---|---|---|---|---|---|
| Bins | Reactivity | (mg) | | TERIA | CoFAR6 | PEPITES |
| Bin 1 | Very High | <100 | mg | 3 | 37 | 29 |
| Bin 2 | High | 100 to <300 | mg | 10 | 13 | 41 |
| Bin 3 | Moderate | 300 to <1000 | mg* | 30 | 34 | 24 |
| Bin 4 | Low | ≥1000 | mg | 25 | | |

| | | | |
|---|---|---|---|
| Total per study & CRD bin | 68 | 84 | 94 |

* Bin 3 includes samples that are greater than 1000 mg (1043 mg and 1044 mg; due to the extra 600 mg dose). Table 7 represents the distribution of the 246 subjects across cohorts and bins. Bin 3 includes subjects with a CRD value of 1043 (CAFETERIA) and 1044 (CoFAR6).

Binning Accuracy: The Test returns a reactivity score and thus assigns a reactivity bin to each patient. To calculate binning accuracy, the assigned bins for the 246 validation samples were compared to the bins into which the samples fell based on the patient's CRD levels. Two types of accuracy can be considered: 2-sided accuracy, namely the proportion of samples that were assigned correctly within ±one bin; and 1-sided accuracy, namely the proportion of samples that are assigned correctly within ±one bin or assigned to a bin with a lower threshold range. One-sided accuracy took into account the impact of returning a reactivity that would have been safe for the patient because a patient mistakenly placed in a bin with a lower reactivity than that of the patient's true CRD bin would unwittingly consume a higher dose than would be safe. Table 8 sets forth the difference between 2-sided and 1-sided accuracy calculations:

TABLE 8

| | | Bin (CRD level) | | | | | | Bin (CRD level) | | | |
|---|---|---|---|---|---|---|---|---|---|---|---|
| | | 1 | 2 | 3 | 4 | | | 1 | 2 | 3 | 4 |
| Assigned | 1 | | | x | X | Assigned | 1 | | | | |
| Bin | 2 | | | | X | Bin | 2 | | | | |
| | 3 | (x) | | | | | 3 | (x) | | | |
| | 4 | (x) | (x) | | | | 4 | (x) | (x) | | |

Table 8 represents possible combinations of bin allocation based on CRD and bin based on predicted reactivity. Patients falling in bins marked with an “x” or “(x)” fall outside of the ±one bin accuracy calculation. The 2-sided accuracy considered any patient not assigned within 1 bin of its CRD-level bin, that is those samples in boxes marked by “x” or “(x)”, to be inaccurately binned. The 1-sided accuracy calculation only considered samples placed in the boxes containing a “(x)” to be inaccurate.

Binning accuracy was calculated using the criteria described in connection with Table 8 for the 246 validation samples and the overall accuracy results can be seen in Table 9:

TABLE 9

| | CAFETERIA | CoFAR6 | PEPITES | Overall |
|---|---|---|---|---|
| 2-sided accuracy | 93% | 75% | 85% | 84% |
| 1-sided accuracy | 93% | 87% | 91% | 90% |

Table 9 represents 2-sided and 1-sided accuracy results for validation patients in each study. Tables 10A to 1° C. shows the exact patient distributions from each of the CAFETERIA, CoFAR6, and PEPITES studies, respectively:

TABLE 10A
CAFETERIA

Bin (CRD level)

| | | 1 | 2 | 3 | 4 |
|---|---|---|---|---|---|
| Assigned Bin | 1 | 0 | 1 | 0 | 0 |
| | 2 | 0 | 0 | 2 | 0 |
| | 3 | 3 | 7 | 21 | 9 |
| | 4 | 0 | 2 | 7 | 16 |

TABLE 10B
CoFAR6

Bin (CRD level)

| | | 1 | 2 | 3 | 4 |
|---|---|---|---|---|---|
| Assigned Bin | 1 | 17 | 4 | 10 | 0 |
| | 2 | 9 | 5 | 5 | 0 |
| | 3 | 8 | 4 | 9 | 0 |
| | 4 | 3 | 0 | 10 | 0 |

TABLE 10C
PEPITES

Bin (CRD level)

| | | 1 | 2 | 3 | 4 |
|---|---|---|---|---|---|
| Assigned Bin | 1 | 11 | 13 | 6 | 0 |
| | 2 | 13 | 10 | 8 | 0 |
| | 3 | 4 | 15 | 7 | 0 |
| | 4 | 1 | 3 | 3 | 0 |

Tables 10A to 1° C. represent distributions of Assigned Bin versus CRD level Bin by study type.

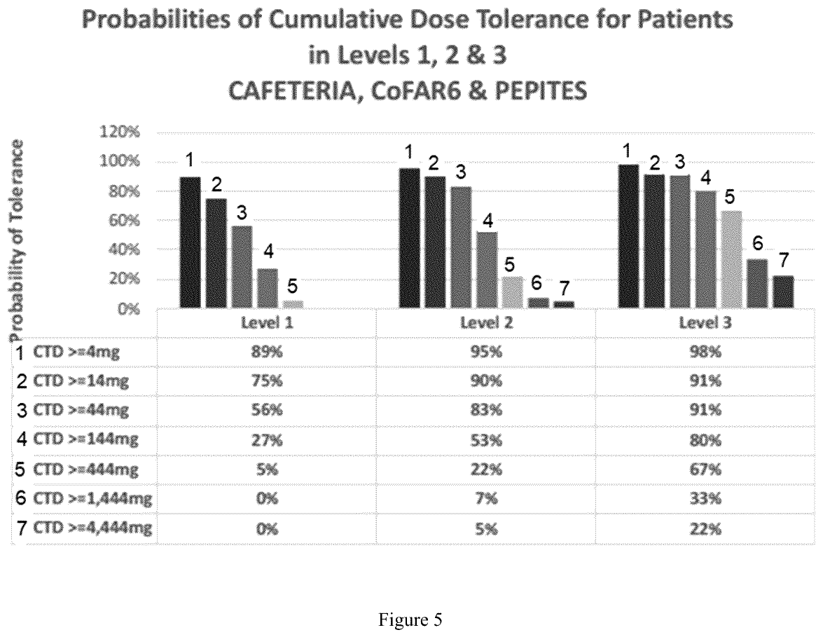

Assignment of Subjects to Levels of Reactivity: In the set of 246 validation samples, the bin assignment accuracy of the test was unexpectedly high: 84% for 2-sided accuracy and 90% for single-sided accuracy. The assignment of these subjects to bins enabled calculation of the probability that a subject would react to a given amount of peanut protein based on the subject's assigned bin number. The levels of peanut protein for which these probabilities were calculated were based on the levels observed in an OFC following PRACTALL guidelines. The probabilities of reaction for the assigned bins can be seen in FIG. 5. Probabilities were calculated using the CRD values of validation samples assigned to each bin. CAFETERIA CRD values, which did not include the first 1 mg dose, have been converted to the CRD levels that would be used in a PRACTALL OFC. For example, a CAFETERIA CRD of 43 mg was considered the same as a CoFAR6 CRD of 44 mg.

The profile of reaction probabilities for assigned bins 1 and 2 showed that, while different, assignment to either bin indicated that a subject was likely to react to low levels of peanut protein and to do so with somewhat similar probabilities. To provide patients with impactful information from the Test, the assigned bins were converted to Reactivity levels. Reactivity Level 1 (high) applied to subjects assigned to bin 1 or 2, Level 2 (moderate) contained bin 3 subjects, and Level 3 (low) contained bin 4 subjects. The probability of reacting to a given cumulative dose if the patient was in Level 1 was, on average, 3.4 times higher than if the patient was placed in Level 3. Level 2 reaction probabilities compared to Level 3 were on average two times higher. FIG. 4, Panel B shows the reaction probabilities for the three levels while Table 11 shows the probability of reaction ratios of Levels 1 and 2 compared to Level 3:

TABLE 11

| | | Level 1: Probability | Level 2: Probability |
|---|---|---|---|
| | | of reaction of Level | of reaction of Level |
| | | 1 subject divided by | 2 subject divided by |
| | | probability of reaction | probability of reaction |
| Cumulative Dose | | of a Level 3 subject | of a Level 3 subject |
| 4 | mg | 4.7 | 2.1 |
| 14 | mg | 2.9 | 1.2 |
| 44 | mg | 4.9 | 1.9 |
| 144 | mg | 3.6 | 2.4 |
| 444 | mg | 2.8 | 2.3 |
| 1444 | mg | 1.6 | 1.4 |

| | | |
|---|---|---|
| Average | 3.4 | 1.9 |

Table 11 shows the ratio of reaction probabilities, at each cumulative dose, for subjects in Level 1 or 2 compared to those in Level 3.

Each reactivity Level has also been profiled in terms of the cumulative tolerated doses (CTD) of its subjects to determine probabilities of tolerating given levels of peanut protein in an OFC. CTD is the sum of the doses that the subject has successfully consumed in an OFC with no reaction; it is one dose lower than the patients CRD. For example, if a subject successfully consumes doses of 1 mg, 3 mg and 10 mg but reacts after the 30 mg dose, their CTD is 14 mg (lmg+3 mg+10 mg) and their CRD is 44 mg (lmg+3 mg+10 mg+30 mg). The proportion of subjects in each Level that can tolerate cumulative doses tested in a PRACTALL OFC can be seen in FIG. 5. In these analyses, subjects in Level 3 (low reactivity) are more likely to tolerate higher cumulative doses of peanut protein than those in Level 1 (high reactivity) or in Level 2 (moderate reactivity). For example, 80% of Level 3 subjects from the CAFETERIA, CoFAR6 and PEPITES cohorts can tolerate a cumulative dose of 144 mg or more but the likelihood of this tolerance falls to 27% for subjects in Level 1.

### Example 3: PRT Reactivity Test Data

Summary: Exemplified are data that combines IgE epitope reactivity levels from a BBEA conducted in accordance with Example 1 into a regression value which was correlated significantly (p-value=0.0093) with clinical data from a PRACTALL dosing protocol peanut oral food challenge study. The samples analyzed originated from the PEPITES clinical trial. It is demonstrated in this Example 3 that peanut allergen epitopes can be used to predict the CRD of an OFC. This finding enables a patient's reactivity level to a peanut allergen to be predicted from epitope reactivity, as measured in a BBEA blood test, as opposed to undergoing a risky and time-consuming OFC.

Data Example: Table 12 sets forth the clinical information for the samples along with the two calibrated IgE epitope levels (NetMFI) of interest acquired by a LUMINEX® instrument running the BBEA from serum samples. The epitopes of interest are “ara h 2.008,” with the alternative names “ara h 2_008” or “h2.008” and having the amino acid sequence WELQGDRRCQSQLER (SEQ ID NO:1), and “ara h 3.100,” with the alternative names “ara h 3_100” or “h3.100” and the amino acid sequence EYDEDEYEYDEEDRR (SEQ ID NO:2). The precise calibration method and whether the sample is serum or plasma were unexpectedly found not to be critical factors in the success of the method. Briefly, the calibration method was to run a known, well-characterized, control sample on each plate along with the clinical samples and the binding data for each epitope in the assay was multiplied by a factor to bring the value of the control sample back to the historically characterized median value. Full details as to how the instrument level data was converted from BBEA LUMINEX® instrument files to the Calibrated NetMFI values follow.

Input Data Description: The required LUMINEX® file is generated by Lumenix's XPONENT® software build 4.3.299.0. There are several sample types that must be present in a LUMENIX® file:
1. Patient samples—Three replicates. Each plate may contain multiple patients.
2. Positive control samples—Three replicates: Every plate has control samples.
3. Serum samples were used with the medium BIOIVT® positive serum as the calibration control labelled BVT-M.
   The LUMINEX® data file has several sections. Each section except the first header section was designated by a row containing the text “DataType:”. The “Net MFI” section contained all the numerical values used in the algorithm processing. The “Net MFI” section had a row per Well Location on a plate. There were 64 analyte values reported for each Well. The two Wells of interest were Analyte 66 and Analyte 95 representing epitopes for h2.008 and h3.100 respectively.

Data Processing: For each Well on a plate extract from the “Net MFI” section, the Analyte 66 and Analyte 95 numerical values were determined and assigned to the appropriate sample and replicate below.
P(epitope)ᵣₙ was the numerical value from the “Net MFI” section for the positive control well reported from the LUMINEX® system for replicate n (1..3) for each epitope (h2.008 and h3.100).
S(epitope)ᵣₙ was the numerical value from the “Net MFI” section for the patient sample well reported from the LUMINEX® system for replicate n (1..3) for each epitope (h2.008 and h3.100).
The Positive Control sample used in the correction factor was labeled BVT-M for Serum samples.

Calculations: The Prediction Score (S) was calculated using Equations (1) to (7):

$$Positive Control NetMFI ( epitope ) = {log}_{2} ( Median ( {P ( epitope )}_{r 1} , {P ( epitope )}_{r 2} , {P ( epitope )}_{r 3} ) + 1 ) \qquad Equation ( 1 )$$

$$MedianNetMFI ( S , Epitope ) = \{ \begin{matrix}MedianNetMFI ( S , Epitope ) , & if MedianNetMFI ( S , Epitope ) \\ 0 , & if MedianNetMFI ( S , Epitope )\end{matrix} \qquad Equation ( 2 )$$

$$NetMFI ( S , epitope ) = {log}_{2} ( MedianNetMFI ( S , epitope ) + 1 ) \qquad Equation ( 3 )$$

$$Correction Factor ( epitope ) = \frac{Constant ( epitope )}{Positive Control Net MFI ( epitope )} \qquad Equation ( 4 )$$

where the constant (epitope value) is an epitope-specific correction constant depending on sample type:

$$Constant ( Serum h 2.008 ) = 6.6865$$

$$Constant ( Serum h 3.1 ) = 7.6439$$

$$Calibrated NetMFI ( S , epitope ) = \{ \begin{matrix}Correction Factor ( epitope ) NetMFI ( S , epitope ) , & if NetMFI ( S , epitope ) > 0 \\ 0 , & if NetMFI ( S , epitope ) \leq 0\end{matrix} \qquad Equation ( 5 )$$

$$Calibrated NetMFI ( S , epitope ) = \{ \begin{matrix}Correction Factor ( epitope ) NetMFI ( S , epitope ) , & if NetMFI ( S , epitope ) \geq LOD ( Epitope ) \\ 0 , & if NetMFI ( S , epitope ) < LOD ( Epitope )\end{matrix} \qquad Equation ( 6 )$$

$$where :$$

$$LOD ( h 2.008 ) = {log}_{2} ( 2.4 + 1 )$$

$$LOD ( h 3.1 ) = {log}_{2} ( 2.4 + 1 )$$

$$Prediction Score ( S ) = Constant ( Alg ) + C 1 ( Alg ) * Calibrated NetMFI ( S , h 2.008 ) + C 2 ( Alg ) * Calibrated NetMFI ( S , h 3.1 ) \qquad Equation ( 7 )$$

Intercept (Alg), Cl(Alg), C₂(Alg) values are obtained from Table 12.

TABLE 12

| Intercept | C1 | C2 |
|---|---|---|
| 4.8542 | 0.0446 | −0.1148 |

The parameters defined in Table 12 were obtained by using standard least squares regression by minimizing the residual of the sum of squared errors given {yᵢ, x_{i,1}, x_{i,2}}, {yᵢ, x_{i,1}, x_{i,2}} are the {log base 2 Cumulative Reactive Dose, Calibrated NetMFI h2.008, Calibrated NetMFI h3.100} values for sample i. These data correspond with those set forth in columns two through four of Table 13.

Regression Algorithm: The regression algorithm is a linear combination of epitopes h2.008 and h3.100 calibrated NetMFI scores as determined by Equations (1) through (7). The output of the algorithm was a single regression value per sample and the actual sample values are set forth in Table 13. When the calculated coefficients are applied, the data set forth in column five of Table 13 are generated:

TABLE 13

| | Cumulative | Calibrated | Calibrated | |
|---|---|---|---|---|
| Sample | Reactive | NetMFI | NetMFI | Regression |
| ID | Dose | ara h2.008 | ara h3.100 | Value |
| DBV061 | 144 | 1.553896555 | 1.579789689 | 4.854211995 |
| DBV120 | 444 | 6.765606856 | 4.444877456 | 4.645991259 |
| DBV122 | 44 | 2.276417308 | 9.295990996 | 3.888822336 |
| DBV139 | 444 | 7.881352726 | 2.798192611 | 4.884794688 |
| DBV140 | 444 | 2.534296126 | 0.996736319 | 4.967321912 |
| DBV001 | 444 | 4.354027715 | 5.303817395 | 4.439769636 |
| DBV020 | 44 | 10.21986553 | 11.52124011 | 3.987938675 |
| DBV034 | 144 | 6.303348942 | 9.806522067 | 4.009952553 |
| DBV065 | 14 | 3.0749308 | 9.113688179 | 3.945385985 |
| DBV077 | 14 | 6.686500527 | 8.131781213 | 4.219279319 |
| DBV080 | 144 | 3.545837611 | 1.034027052 | 5.012468717 |
| DBV099 | 144 | 7.559245298 | 1.638894102 | 5.191593882 |
| DBV158 | 444 | 3.67450387 | 1.034027052 | 5.01821131 |
| DBV064 | 144 | 1.995839321 | 7.561684557 | 4.075362644 |
| DBV100 | 144 | 1.319178035 | 5.289995285 | 4.24702852 |
| DBV117 | 144 | 3.991678641 | 1.575452356 | 5.032367356 |
| DBV118 | 14 | 7.007758902 | 7.795294412 | 4.272239439 |
| DBV142 | 44 | 2.579584901 | 10.38439829 | 3.777426278 |
| DBV145 | 44 | 4.312937016 | 6.132904012 | 4.342773469 |
| DBV153 | 444 | 6.353078101 | 5.056936877 | 4.557327476 |
| DBV166 | 144 | 5.54303342 | 2.307995985 | 4.836696114 |
| DBV007 | 44 | 3.049748178 | 4.433668074 | 4.481432817 |
| DBV023 | 4 | 5.481736744 | 6.755211417 | 4.323510789 |
| DBV067 | 144 | 5.582922991 | 2.381959681 | 4.82998693 |
| DBV073 | 444 | 7.424897342 | 7.714050408 | 4.300182185 |
| DBV108 | 144 | 5.105652362 | 8.299275778 | 4.129498438 |
| DBV127 | 14 | 6.004842943 | 2.051708393 | 4.886724037 |
| DBV138 | 144 | 8.059242131 | 8.678143428 | 4.217835866 |
| DBV003 | 144 | 1.584962501 | 4.247927513 | 4.366636629 |
| DBV009 | 1 | 8.820178962 | 10.08281434 | 4.090570215 |
| DBV040 | 444 | 4.64385619 | 2.807354922 | 4.739248103 |
| DBV070 | 144 | 10.28019079 | 8.951284715 | 4.285609513 |
| DBV141 | 144 | 1.584962501 | 1 | 4.854211995 |
| DBV058 | 444 | 4.594634276 | 1.003827637 | 5.059278284 |
| DBV063 | 144 | 5.470948047 | 1.591029162 | 5.098389648 |
| DBV115 | 444 | 1.588305909 | 2.594856799 | 4.556375403 |
| DBV131 | 444 | 3.176611818 | 3.714603658 | 4.569628872 |
| DBV146 | 14 | 1.588305909 | 1.003827637 | 4.854211995 |
| DBV154 | 144 | 6.473057503 | 8.988469724 | 4.111422642 |
| DBV008 | 144 | 8.021995935 | 8.401142367 | 4.247967573 |
| DBV022 | 14 | 6.365902722 | 7.211451712 | 4.310605537 |
| DBV043 | 14 | 5.591508418 | 1.579060712 | 5.103770461 |
| DBV110 | 144 | 7.110048889 | 5.406059438 | 4.551040218 |
| DBV116 | 1 | 6.3047559 | 3.510441571 | 4.732676867 |
| DBV151 | 444 | 8.538218671 | 9.343373704 | 4.162858542 |
| DBV028 | 44 | 8.496142998 | 6.671832578 | 4.467619021 |
| DBV033 | 144 | 8.548841561 | 4.61534104 | 4.706014296 |
| DBV039 | 444 | 6.14462665 | 5.898658404 | 4.451411486 |
| DBV059 | 144 | 7.566367787 | 8.766790414 | 4.185663187 |
| DBV111 | 144 | 7.675958749 | 9.673147075 | 4.086523174 |
| DBV136 | 444 | 9.13050615 | 7.083372003 | 4.448695395 |
| DBV160 | 44 | 7.813963658 | 4.851359977 | 4.646125298 |
| DBV021 | 144 | 5.591508418 | 6.126395897 | 4.40058527 |
| DBV081 | 4 | 1.991735485 | 9.106843987 | 3.897826721 |
| DBV128 | 444 | 8.218563295 | 6.282441561 | 4.49992431 |
| DBV162 | 444 | 1.578413028 | 1.321305418 | 4.854211995 |
| DBV018 | 444 | 1.613102818 | 1.618856715 | 4.854211995 |
| DBV055 | 14 | 5.910461974 | 2.042769736 | 4.88353763 |
| DBV071 | 144 | 10.05965965 | 6.952929308 | 4.505137244 |
| DBV124 | 144 | 5.088772818 | 5.320856838 | 4.470606771 |
| DBV019 | 44 | 8.996903466 | 8.052054236 | 4.331547534 |
| DBV030 | 14 | 7.631717044 | 9.524733843 | 4.101583401 |
| DBV069 | 144 | 8.411144598 | 10.41332113 | 4.034378888 |
| DBV107 | 14 | 7.31654443 | 7.471919354 | 4.323137914 |
| DBV006 | 144 | 3.545837611 | 1.03342769 | 5.012468717 |
| DBV083 | 4 | 7.338702965 | 9.078284892 | 4.139748914 |
| DBV101 | 14 | 1.624550003 | 3.575072425 | 4.443866658 |
| DBV157 | 144 | 8.485219737 | 2.901198311 | 4.899923342 |
| DBV159 | 144 | 2.379922738 | 7.791175472 | 4.066164063 |
| DBV075 | 444 | 4.962157393 | 3.146632105 | 4.714512328 |
| DBV084 | 444 | 7.799535657 | 4.360042277 | 4.701874595 |
| DBV085 | 444 | 10.44827369 | 7.432818707 | 4.467400238 |
| DBV098 | 144 | 2.531931496 | 1.573316053 | 4.967216375 |
| DBV161 | 144 | 1.552446689 | 6.428149155 | 4.116391689 |
| DBV057 | 144 | 2.601554373 | 6.235149539 | 4.254655872 |
| DBV093 | 144 | 6.335023479 | 8.929663922 | 4.112011657 |
| DBV137 | 4 | 2.012837225 | 7.632012158 | 4.068049116 |
| DBV156 | 144 | 3.724191406 | 1.606973102 | 5.020428948 |
| DBV005 | 144 | 1.578413028 | 5.409616543 | 4.233298441 |
| DBV013 | 144 | 9.099234713 | 7.736116009 | 4.37237801 |
| DBV048 | 4 | 5.858334454 | 2.561375778 | 4.821685704 |
| DBV123 | 444 | 5.500737176 | 1.570500368 | 5.099719187 |
| DBV129 | 144 | 7.273599646 | 7.290417359 | 4.34205394 |
| DBV147 | 144 | 6.424553047 | 0.99087541 | 5.14095065 |
| DBV004 | 444 | 8.046361657 | 9.871905238 | 4.080241501 |
| DBV015 | 144 | 0.996890191 | 1.584962501 | 4.854211995 |
| DBV046 | 144 | 1.58003357 | 5 | 4.280314059 |
| DBV089 | 444 | 6.284177195 | 2.321928095 | 4.868175491 |
| DBV092 | 144 | 7.870266389 | 4.321928095 | 4.709406157 |
| DBV104 | 44 | 6.972602335 | 2.584962501 | 4.868710087 |
| DBV113 | 144 | 4.445563639 | 1.584962501 | 5.05262501 |
| DBV134 | 4 | 4.740100711 | 2.807354922 | 4.743543658 |
| DBV036 | 444 | 3.20432879 | 1.347311452 | 4.997226602 |
| DBV155 | 44 | 6.780155355 | 5.001111572 | 4.582796261 |

Results: Analysis of the data set forth in this Example 3 results in a Spearman’s rank correlation coefficient of 0.2668 with a significance p-value of 0.0093.

### Example 4: Personalized Severity Risk

An example of the use of an assay, such as the BBEA method, that combines specific peanut protein epitopes and clinical information on severe reaction for the prediction of personalized severe reaction risk for patients is provided.

There are two main components of the methodology. First, a peanut threshold examination, as described herein, is performed that places the patient into one of three levels of epitope reactivity to epitopes h2.008 (WELQGDRRCQSQLER) and h3.100 (EYDEDEYEYDEEDRR). Levels 1, 2, and 3 are described herein. Second, a severe reaction database is generated. This is a database of patients who have undergone standardized (e.g., PRACTALL) oral food challenges (OFCs) to determine anaphylactic risk. Specifically, the database records at which amount of ingested peanut each patient reached a state of severe reaction and also the results of the peanut threshold test for a particular patient. This allows the determination of the percentage of patients in each Level (1, 2, or 3) reach a severe reaction. Any standardized OFCs database can be used, and indeed, the database can be extended and made more generalizable by incorporating more patient data.

The methodology is used to determine a new patient's risk of severe reaction. For example, blood is sampled from the patient and assessed by the previously described peanut threshold test that places the patient into one of three levels of epitope reactivity to epitopes h2.008 and h3.100. Depending on the patient's level, the percentage of patients in the reactivity database with the same assigned level and experiencing a severe reaction is determined and reported out.

A reactivity database was generated from the boiled peanut oral immunotherapy for the treatment of peanut allergy (BOPI) cohort to demonstrate the creation of such a database and the resultant risks of severe reaction for each of the three levels of epitope reactivity. Table 14 summarizes the generation of a reactivity database.

TABLE 14
A reactivity database

| | CRD ≤30 | | CRD ≤43 | | CRD ≤143 | |
|---|---|---|---|---|---|---|
| | # | % | # | % | # | % |
| Level | Patients | anaphylaxis | Patients | anaphylaxis | Patients | anaphylaxis |
| Level 1 | 10 | 20% | 22 | 18% | 29 | 28% |
| Level 2 | 1 | 0% | 6 | 0% | 11 | 9% |
| Level 3 | 0 | 0% | 1 | 0% | 3 | 0% |

| | CRD ≤443 | | CRD ≤4443 | |
|---|---|---|---|---|
| | # | % | # | % |
| Level | Patients | anaphylaxis | Patients | anaphylaxis |
| Level 1 | 30 | 30% | 30 | 30% |
| Level 2 | 18 | 17% | 21 | 19% |
| Level 3 | 6 | 0% | 12 | 33% |

For example, a new patient assigned to Level 1 would have a 20% chance of reaching anaphylaxis after consuming just 30 mg (CRD) of peanut. In contrast, a new patient assigned to Level 3 would be unlikely to reach anaphylaxis until consuming over 443 mg (CRD) of peanut.

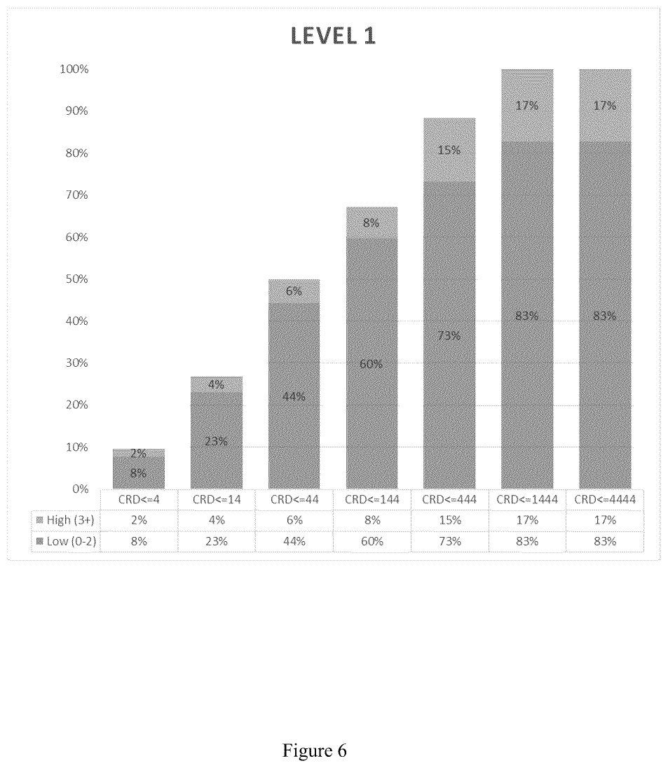

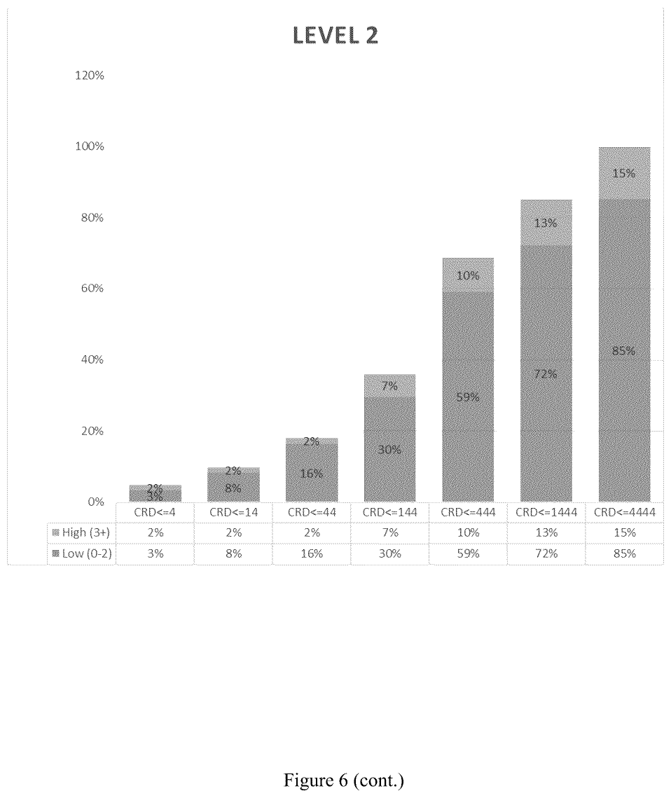

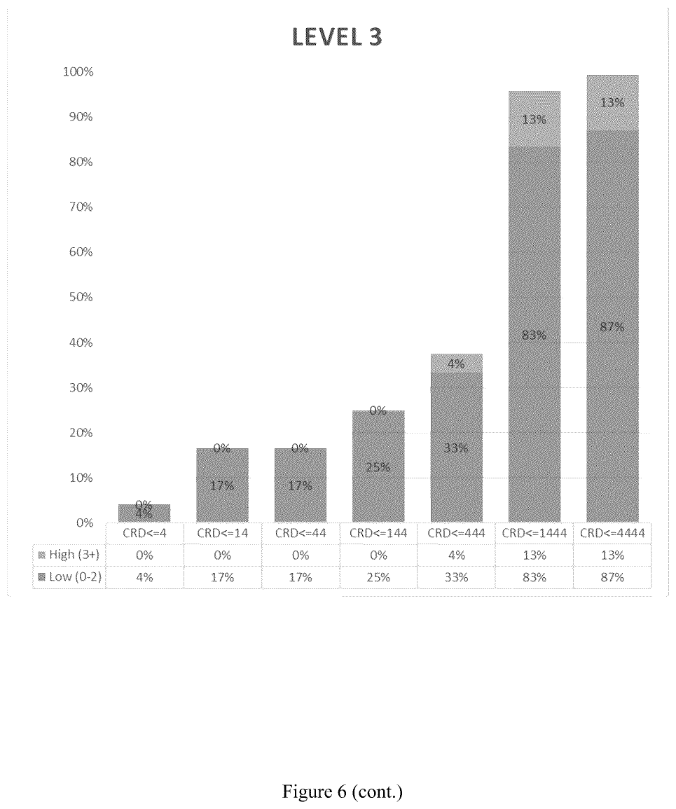

FIG. 6 shows a representative severe reaction risks by level. For a subject in Level 1: i) the subject has a 2% risk of a Cofar grade 3 or higher reaction vs an 8% risk of a Cofar grade 2 or lower reaction after consuming ≤4 mg (CRD) of peanut; ii) the subject has a 4% risk of a Cofar grade 3 or higher reaction vs a 23% risk of a Cofar grade 2 or lower reaction after consuming ≤14 mg (CRD) of peanut; iii) the subject has a 6% risk of a Cofar grade 3 or higher reaction vs a 44% risk of a Cofar grade 2 or lower reaction after consuming ≤44 mg (CRD) of peanut; iv) the subject has an 8% risk of a Cofar grade 3 or higher reaction vs a 60% risk of a Cofar grade 2 or lower reaction after consuming ≤144 mg (CRD) of peanut; v) the subject has a 15% risk of a Cofar grade 3 or higher reaction vs a 73% risk of a Cofar grade 2 or lower reaction after consuming ≤444 mg (CRD) of peanut; vi) the subject has a 17% risk of a Cofar grade 3 or higher reaction vs an 83% risk of a Cofar grade 2 or lower reaction after consuming ≤1444 mg (CRD) of peanut; and vii) the subject has a 17% risk of a Cofar grade 3 or higher reaction vs an 83% risk of a Cofar grade 2 or lower reaction after consuming ≤4444 mg (CRD) of peanut.

For a subject in Level 2: i) the subject has a 2% risk of a Cofar grade 3 or higher reaction vs a 3% risk of a Cofar grade 2 or lower reaction after consuming ≤4 mg (CRD) of peanut; ii) the subject has a 2% risk of a Cofar grade 3 or higher reaction vs an 8% risk of a Cofar grade 2 or lower reaction after consuming ≤14 mg (CRD) of peanut; iii) the subject has a 2% risk of a Cofar grade 3 or higher reaction vs a 16% risk of a Cofar grade 2 or lower reaction after consuming ≤44 mg (CRD) of peanut; iv) the subject has a 7% risk of a Cofar grade 3 or higher reaction vs a 30% risk of a Cofar grade 2 or lower reaction after consuming ≤144 mg (CRD) of peanut; v) the subject has a 10% risk of a Cofar grade 3 or higher reaction vs a 59% risk of a Cofar grade 2 or lower reaction after consuming ≤444 mg (CRD) of peanut; vi) the subject has a 13% risk of a Cofar grade 3 or higher reaction vs a 72% risk of a Cofar grade 2 or lower reaction after consuming ≤1444 mg (CRD) of peanut; and vii) the subject has a 15% risk of a Cofar grade 3 or higher reaction vs an 85% risk of a Cofar grade 2 or lower reaction after consuming ≤4444 mg (CRD) of peanut.

For a subject in Level 3: i) the subject has a 0% risk of a Cofar grade 3 or higher reaction vs a 4% risk of a Cofar grade 2 or lower reaction after consuming ≤4 mg (CRD) of peanut; ii) the subject has a 0% risk of a Cofar grade 3 or higher reaction vs a 17% risk of a Cofar grade 2 or lower reaction after consuming ≤14 mg (CRD) of peanut; iii) the subject has a 0% risk of a Cofar grade 3 or higher reaction vs a 17% risk of a Cofar grade 2 or lower reaction after consuming ≤44 mg (CRD) of peanut; iv) the subject has a 0% risk of a Cofar grade 3 or higher reaction vs a 25% risk of a Cofar grade 2 or lower reaction after consuming ≤144 mg (CRD) of peanut; v) the subject has a 4% risk of a Cofar grade 3 or higher reaction vs a 33% risk of a Cofar grade 2 or lower reaction after consuming ≤444 mg (CRD) of peanut; vi) the subject has a 13% risk of a Cofar grade 3 or higher reaction vs an 83% risk of a Cofar grade 2 or lower reaction after consuming ≤1444 mg (CRD) of peanut; and vii) the subject has a 13% risk of a Cofar grade 3 or higher reaction vs an 87% risk of a Cofar grade 2 or lower reaction after consuming ≤4444 mg (CRD) of peanut.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

1. A method for determining the risk of anaphylaxis in a subject allergic to peanuts, the method comprising:
a) determining a threshold cumulative reactive dose of a peanut peptide for the subject comprising:
contacting at least one first peanut peptide comprising the amino acid sequence WELQGDRRCQSQLER (SEQ ID NO:1), or an amino acid sequence comprising SEQ ID NO:1 but having one to four conservative amino acid substitutions therein, coupled to at least one first solid support with at least one first biological sample obtained from the subject, wherein the contacting is under conditions sufficient to permit binding of at least one allergy associated immunoglobulin (AAI-1) present in the at least one first biological sample to the at least one first peanut peptide to form at least one AAI-1-peptide-solid support complex;
contacting at least one second peanut peptide comprising the amino acid sequence EYDEDEYEYDEEDRR (SEQ ID NO:2), or an amino acid sequence comprising SEQ ID NO:2 but having one to four conservative amino acid substitutions therein, coupled to at least one second solid support with at least one second biological sample obtained from the subject, wherein the contacting is under conditions sufficient to permit binding of at least one second allergy associated immunoglobulin (AAI-2) present in the at least one second biological sample to the at least one second peanut peptide to form at least one AAI-2-peptide-solid support complex;
contacting the at least one AAI-1-peptide-solid support complex with at least one AAI-1-specific labeling reagent under conditions sufficient to permit binding of the at least one AAI-1 specific labeling reagent to the at least one AAI-1-peptide-solid support complex;
measuring the binding of the at least one AAI-1-specific labeling reagent to the at least one AAI-1-peptide-solid support complex, thereby determining at least one AAI-1-peptide binding value;
contacting the at least one AAI-2-peptide-solid support complex with at least one AAI-2-specific labeling reagent under conditions sufficient to permit binding of the at least one AAI-2 specific labeling reagent to the at least one AAI-2-peptide-solid support complex;
measuring the binding of the at least one AAI-2-specific labeling reagent to the at least one AAI-2-peptide-solid support complex, thereby determining at least one AAI-2-peptide binding value;
wherein:
when the at least one AAI-1 peptide binding value and the at least one AAI-2 peptide binding value are combined to generate a combined peptide binding value and the combined peptide binding value is greater than a first threshold value, the subject has a cumulative reactive dose of less than a first amount of a peanut protein, and the subject is designated in Level 1;
when the at least one AAI-1 peptide binding value and the at least one AAI-2 peptide binding value are combined to generate a combined peptide binding value and the combined peptide binding value is less than or equal to the first threshold value but greater than a second threshold value, the subject has a cumulative reactive dose of greater than or equal to about the first amount of a peanut protein to less than a second amount of a peanut protein, and the subject is designated in Level 2; and
when the at least one AAI-1 peptide binding value and the at least one AAI-2 peptide binding value are combined to generate a combined peptide binding value and the combined peptide binding value is less than or equal to a second threshold value, the subject has a cumulative reactive dose of greater than or equal to the second amount of a peanut protein, and the subject is designated in Level 3; and
b) determining the risk of anaphylaxis of the subject, wherein:
for a subject in Level 1: i) the subject has a 2% risk of a Cofar grade 3 or higher reaction vs an 8% risk of a Cofar grade 2 or lower reaction after consuming ≤4 mg (CRD) of peanut; ii) the subject has a 4% risk of a Cofar grade 3 or higher reaction vs a 23% risk of a Cofar grade 2 or lower reaction after consuming 14 mg (CRD) of peanut; iii) the subject has a 6% risk of a Cofar grade 3 or higher reaction vs a 44% risk of a Cofar grade 2 or lower reaction after consuming 44 mg (CRD) of peanut; iv) the subject has an 8% risk of a Cofar grade 3 or higher reaction vs a 60% risk of a Cofar grade 2 or lower reaction after consuming 144 mg (CRD) of peanut; v) the subject has a 15% risk of a Cofar grade 3 or higher reaction vs a 73% risk of a Cofar grade 2 or lower reaction after consuming 444 mg (CRD) of peanut; vi) the subject has a 17% risk of a Cofar grade 3 or higher reaction vs an 83% risk of a Cofar grade 2 or lower reaction after consuming 1444 mg (CRD) of peanut; and vii) the subject has a 17% risk of a Cofar grade 3 or higher reaction vs an 83% risk of a Cofar grade 2 or lower reaction after consuming 4444 mg (CRD) of peanut;
for a subject in Level 2: i) the subject has a 2% risk of a Cofar grade 3 or higher reaction vs a 3% risk of a Cofar grade 2 or lower reaction after consuming ≤4 mg (CRD) of peanut; ii) the subject has a 2% risk of a Cofar grade 3 or higher reaction vs an 8% risk of a Cofar grade 2 or lower reaction after consuming 14 mg (CRD) of peanut; iii) the subject has a 2% risk of a Cofar grade 3 or higher reaction vs a 16% risk of a Cofar grade 2 or lower reaction after consuming 44 mg (CRD) of peanut; iv) the subject has a 7% risk of a Cofar grade 3 or higher reaction vs a 30% risk of a Cofar grade 2 or lower reaction after consuming 144 mg (CRD) of peanut; v) the subject has a 10% risk of a Cofar grade 3 or higher reaction vs a 59% risk of a Cofar grade 2 or lower reaction after consuming 444 mg (CRD) of peanut; vi) the subject has a 13% risk of a Cofar grade 3 or higher reaction vs a 72% risk of a Cofar grade 2 or lower reaction after consuming 1444 mg (CRD) of peanut; and vii) the subject has a 15% risk of a Cofar grade 3 or higher reaction vs an 85% risk of a Cofar grade 2 or lower reaction after consuming 4444 mg (CRD) of peanut; and
for a subject in Level 3: i) the subject has a 0% risk of a Cofar grade 3 or higher reaction vs a 4% risk of a Cofar grade 2 or lower reaction after consuming ≤4 mg (CRD) of peanut; ii) the subject has a 0% risk of a Cofar grade 3 or higher reaction vs a 17% risk of a Cofar grade 2 or lower reaction after consuming 14 mg (CRD) of peanut; iii) the subject has a 0% risk of a Cofar grade 3 or higher reaction vs a 17% risk of a Cofar grade 2 or lower reaction after consuming 44 mg (CRD) of peanut; iv) the subject has a 0% risk of a Cofar grade 3 or higher reaction vs a 25% risk of a Cofar grade 2 or lower reaction after consuming 144 mg (CRD) of peanut; v) the subject has a 4% risk of a Cofar grade 3 or higher reaction vs a 33% risk of a Cofar grade 2 or lower reaction after consuming 444 mg (CRD) of peanut; vi) the subject has a 13% risk of a Cofar grade 3 or higher reaction vs an 83% risk of a Cofar grade 2 or lower reaction after consuming 1444 mg (CRD) of peanut; and vii) the subject has a 13% risk of a Cofar grade 3 or higher reaction vs an 87% risk of a Cofar grade 2 or lower reaction after consuming 4444 mg (CRD) of peanut.

2. The method according to claim 1, wherein the first amount of a peanut protein is about 300 mg or about 444 mg of a peanut protein.

3. The method according to claim 1, wherein the second amount of a peanut protein about 1000 mg or about 1444 mg of a peanut protein.

4. The method according to claim 1, wherein the at least one first biological sample and the at least one second biological sample are derived from the same biological sample.

5. The method according to claim 1, wherein the at least one first peanut peptide comprises the amino acid sequence according to SEQ ID NO:1.

6. The method according to claim 1, wherein the at least one second peanut peptide comprises the amino acid sequence according to SEQ ID NO:2.

7. The method according to claim 1, wherein the at least one first solid support comprises at least one first microsphere bead, at least one first glass array, at least one first silicone array, at least one first membrane, or at least one first microtiter plate.

8. The method according to claim 1, wherein the at least one second solid support comprises at least one second microsphere bead, at least second one glass array, at least one second silicone array, at least one second membrane, or at least one second microtiter plate.

9. The method according to claim 8, wherein the at least one first microsphere bead and the at least one second microsphere bead are the same microsphere bead, the at least first one glass array and the at least second one glass array, the at least one first silicone array and the at least one second silicone array are the same silicone array, the at least one first membrane and the at least one second membrane are the same membrane, or the at least one first microtiter plate and the at least one second microtiter plate are the same microtiter plate.

10. The method according to claim 1, wherein the AAI-1 comprises at least one first IgG and/or at least one first IgE.

11. The method according to claim 1, wherein the AAI-2 comprises at least one IgG and/or at least one IgE.

12. The method according to claim 1, wherein the at least one AAI-1-specific labeling reagent comprises at least one first detectably labeled anti-human antibody.

13. The method according to claim 1, wherein the at least one AAI-2-specific labeling reagent comprises at least one second detectably labeled anti-human antibody.

14. The method according to claim 12, wherein the detectable label of the at least one first detectably labeled anti-human antibody comprises at least one first fluorophore.

15. The method according to claim 13, wherein the detectable label of the at least one second detectably labeled anti-human antibody comprises at least one second fluorophore.

16. The method according to claim 15, wherein the at least one first detectably labeled anti-human antibody and the at least one second detectably labeled anti-human antibody have the same chemical structure.

17. The method according to claim 1, wherein the measuring of the binding of the at least one first AAI-1-specific labeling reagent to the at least one AAI-1-peptide-solid support complex is carried out by at least one first point of care device.

18. The method according to claim 1, wherein the measuring of the binding of the at least one AAI-2-specific labeling reagent to the at least one AAI-2-peptide-solid support complex is carried out by at least one second point of care device.

19. The method according to claim 18, wherein the at least one first point of care device and at least one second point of care device are the same point of care device.
